(19) United States
(12) Patent Application Publication
(10) Pub. No.: US 2015/0037293 A1
(43) Pub. Date: Feb. 5, 2015

(54) TECHNOLOGIES FOR PANCREATIC ISLET TRANSPLANTATION

(71) Applicant: THE UAB RESEARCH FOUNDATION, Birmingham, AL (US)

(72) Inventors: Ho-Wook Jun, Birmingham, AL (US); Dong-Jin Lim, Birmingham, AL (US); Patrick TJ Hwang, Birmingham, AL (US)

(21) Appl. No.: 14/383,406

(22) PCT Filed: Mar. 6, 2013
(86) PCT No.: PCT/US13/29315

§ 371 Date: Sep. 5, 2014

Related U.S. Application Data
(60) Provisional application No. 61/607,408, filed on Mar. 6, 2012.
(60) Provisional application No. 61/607,678, filed on Mar. 7, 2012.

(51) Int. Cl. A61L 27/22; D01D 5/00; A61L 27/56; A61L 27/54; A61L 27/38

(52) U.S. Cl. CPC A61L 27/227; A61L 27/54; A61L 27/3804; A61L 27/56; D01D 5/0007; A61L 2430/34; A61L 2400/18; A61L 2300/414; A61L 2300/412; D10B 2331/041; D10B 2401/10
USPC 424/93.7; 514/773; 514/9.1; 435/177; 264/466

(57) ABSTRACT

Biocompatible nanomatrices composed of peptide amphiphiles are provided for the embedding of cell populations for their implantation into a recipient animal or human. To confine the nanomatrix to a site of implantation, the nanomatrix can be encapsulated in a nanofiber sack formed from an electrospun nanofiber sheet. The nanofiber sheets are porous and have surface indentations that promote the vascularization of the implant, thereby maintain the viability and biofunctions of the cells, as wells as delivering cell-product products to the circulatory system to the benefit of the recipient subject. The implants may further include cell growth factors that can be beneficial to the survival of the cells as to promote angiogenesis and infiltration of the implant by new blood vessels.

### CROSS-REFERENCE TO RELATED APPLICATIONS

This application claims priority to U.S. Provisional Patent Application Ser. No. 61/607,408 entitled “TECHNOLOGIES FOR PANCREATIC ISLET TRANSPLANTATION” filed Mar. 6, 2012, and to U.S. Provisional Patent Application Ser. No. 61/607,678 entitled “TECHNOLOGIES FOR PANCREATIC ISLET TRANSPLANTATION” filed Mar. 7, 2012, the entireties of which are hereby incorporated by reference.

### STATEMENT REGARDING FEDERALLY SPONSORED RESEARCH OR DEVELOPMENT

This invention was made with government support under Grant Nos. T32 NIBIB #EB004312-01, DK 52194 and AI 44458 awarded by the National Institutes of Health of the United States government. The government has certain rights in the invention.

### SEQUENCE LISTING

The present disclosure includes a sequence listing incorporated herein by reference in its entirety.

### TECHNICAL FIELD

The present disclosure is generally related to novel biocompatible implants comprising a peptide amphiphile nanomatrix having isolated cells or cell aggregates embedded therein and a surrounding electrospun nanofiber sack. The disclosure further relates to porous electrospun nanofiber sheets having crater-like surface indentations suitable for promoting vascularization of an implant, and to methods of manufacture thereof.

### BACKGROUND

Treatments for diabetes have developed significantly, but the global incidence of diabetes is still increasing, with the total number of people with diabetes estimated to become 366 million in 2030 (Roep et al., (1999) Diabetes 48: 484-490). Type 1 diabetes has a significant societal impact through long-term complications including kidney failure, blindness, nerve damage, and cardiovascular problems. In an attempt to treat type 1 diabetes, pancreatic islet transplantation (PIT) has been tried with consistent and sustained success (Merani et al., (2008) Brit. J. Surgery 95: 1449-1461; Rajab A. Curr. Diabetes Rpts. 10: 332-337). However, a major drawback is the necessity for multiple islet infusions from multiple organs to achieve insulin independence and limited islet graft survival. Only about 10% of recipients maintained insulin independence after 5-year post-islet transplantation (Ryan et al., (2005) Diabetes 54: 2060-2069). Therefore, there remains a significant need to develop an innovative strategy to increase the efficacy of PIT.

Two major factors to be considered for an innovative strategy to increase practical implementation of PIT are recovery of the islet microenvironment to prevent substantial loss caused by disruption of the microenvironment during the peritransplant period, and identifying an alternative islet transplantation site with enhanced revascularization to overcome limitations of current intrahepatic implantation sites.

The ongoing investigation of treatment options for human type I diabetes mellitus requires the continual development of innovative strategies that more effectively restore long-term physiological function, while still maintaining a simple approach with minimal invasiveness. In the past decade, islet engraftment has been heavily investigated as one such promising treatment for type I diabetic patients, offering a less invasive alternative to full pancreas replacement. In spite of the numerous efforts to improve islet engraftment, clinical efficacy is still lacking because the primary focus has been on avoiding host immune response via the development of semipermeable and biocompatible membranes, while relatively ignoring the potential benefits of a more biomimetic engraftment material. The need for improving the biomimetic character of islet scaffolds is supported by recent literature demonstrating that substantial β-cell loss during the peritransplant period is detrimental to the efficacy of islet transplantation. Specifically, the loss of β-cell function during islet isolation is believed to occur from the destruction of the native islet microenvironment, thereby triggering islet death. In addition, the disruption of islet-extracellular matrix (ECM) interactions exposes the islets to a variety of cellular stresses that further contribute to loss of biological functions. Therefore, biomimetic materials that create more ECM-mimicking environments are needed to better maintain islet function and survival during the intermediate stage between implantation and fully restored host integration.

### SUMMARY

Briefly described, one aspect of the disclosure, therefore, encompasses embodiments of a biocompatible implant comprising: (i) a biocompatible nanomatrix gel comprising a plurality of a peptide amphiphile monomers cross-linked by divalent metal anions; and (ii) a biocompatible nanofiber sack, wherein said nanofiber sack is formed from a porous electrospun nanofiber sheet having crater-like surface indentations.

In embodiments of this aspect of the disclosure, the peptide amphiphile monomers can have the formula (CH₃(CH₂)₁₄CONH-GTAGLIGQERGDS) (SEQ ID NO.: 1).

In embodiments of this aspect of the disclosure, the biocompatible implant can further comprise at least one cell growth factor, wherein the at least one cell growth factor can be incorporated in the nanomatrix gel, incorporated in the nanofiber sack, or both incorporated in the nanomatrix gel and in the nanofiber sack.

In embodiments of this aspect of the disclosure, the biocompatible implant can further comprise a population of isolated animal or human cells embedded in the nanomatrix gel.

In embodiments of this aspect of the disclosure, the at least one cell growth factor can be releasable from the biocompatible implant.

In embodiments of this aspect of the disclosure, the at least one cell growth factor can be an angiogenic factor that can induce the formation of a blood vessel when the biocompatible implant is implanted in a recipient animal or human subject.

In embodiments of this aspect of the disclosure, the population of isolated animal or human cells embedded in the gel can be a pancreatic islet or a population of pancreatic islets.

In embodiments of this aspect of the disclosure, the pancreatic islet or islets can be isolated from an animal or human, or a cultured islet or islets.

In embodiments of this aspect of the disclosure, the polymer nanofibers forming the nanofiber sheet can comprise poly-ε-caprolactone.

In embodiments of this aspect of the disclosure, the nanofiber sheet can further comprise at least one cell growth factor.

In embodiments of this aspect of the disclosure, the at least one cell growth factor embedded in the nanofiber sheet, attached to an outer surface thereof, or both embedded in the nanofiber sheet and attached to an outer surface thereof.

In embodiments of this aspect of the disclosure, the at least one cell growth factor is releasable from the implant in a multi-step process.

Another aspect of the disclosure encompasses embodiments of a biocompatible electrospun nanofiber sheet, wherein said sheet is porous and comprises a plurality of crater-like indentations on at least one surface of said nanofiber sheet.

In embodiments of this aspect of the disclosure, the polymer nanofibers forming the nanofiber sheet can comprise poly-ε-caprolactone.

In embodiments of this aspect of the disclosure, the biocompatible nanofiber sheet can further comprise at least one cell growth factor.

In embodiments of this aspect of the disclosure, the at least one cell growth factor can be embedded in the nanofiber sheet, attached to an outer surface thereof, or both embedded in the nanofiber sheet and attached to an outer surface thereof.

In embodiments of this aspect of the disclosure, the the at least one cell growth factor can be releasable from the nanofiber sack.

In some embodiments of this aspect of the disclosure, at least one cell growth factor is an angiogenic factor that can induce the formation of a blood vessel when the biocompatible implant is implanted in a recipient animal or human subject.

Still another aspect of the disclosure encompasses embodiments of a method of manufacturing a biocompatible nanofiber sheet, the method comprising the steps of: (i) electrospinning a biocompatible polymer onto a collector to form a nanofiber sheet, wherein the biocompatible polymer is co-delivered to the collector with a plurality of leachable particles; and (ii) contacting the electrospun nanofiber sheet with a composition capable of removing the particles from the nanofiber sheet, thereby generating a porous nanofiber sheet having crater-like indentations in at least one surface of the nanofiber sheet.

In embodiments of this aspect of the disclosure, the biocompatible polymer can be poly-ε-caprolactone.

In embodiments of this aspect of the disclosure, the biocompatible polymer can be delivered to the collector at a flow rate from about 0.5 ml/h to about 5.0 ml/h, at a distance from about 10 cm to about 30 cm, a voltage from about 10 to about 25 kV and for about 30 min to about 3 h.

In embodiments of this aspect of the disclosure, the method can further comprise the step of contacting the nanofiber sheet with at least one cell growth factor desired to be incorporated into the nanofiber sheet.

In some embodiments of this aspect of the disclosure, the leachable particles can be particles of a carbonate or a bicarbonate, and wherein the composition capable of removing the particles from the nanofiber sheet is an acid, thereby generating a gas that generates the crater-like indentations.

Still yet another aspect of the disclosure encompasses embodiments of a method of maintaining a population of isolated animal cells in a state suitable for implantation into a recipient animal or human subject, the method comprising the steps of (i) embedding a population of cells or cell aggregates thereof, in an implantable biomimetic nanomatrix gel comprising: (a) a plurality of a peptide amphiphile monomers cross-linked by divalent metal anions; and (b) at least one cell growth factor; (ii) encapsulating the nanomatrix gel in a nanofiber sack, wherein said nanofiber sack is formed from a nanofiber sheet manufactured by electrospinning a biocompatible polymer; and (iii) maintaining the encapsulated nanomatrix under conditions substantially allowing the population of cells or cell aggregates thereof to retain viability and their biological function.

In embodiments of this aspect of the disclosure, the cell aggregates are pancreatic islets.

In embodiments of this aspect of the disclosure, the biocompatible polymer forming the nanofiber sheet is poly-ε-caprolactone.

In embodiments of this aspect of the disclosure, the nanofiber sack is porous and includes a plurality of crater-like indentations in an outer surface of the nanofiber sack.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates a strategy using the biomimetic nanomatrix that enhances the efficacy of islet engraftment by improving islet graft survival.

FIGS. 2A and 2B are digital SEM images of an ePCL nanofiber sheet generated by a traditional flat-sheet method (FIG. 2A) and an ePCL nanofiber sheet incorporating crater-like structures (FIG. 2B) generated according to the methods of the disclosure.

FIG. 3 is a graph illustrating multi-stage FGF-1 release kinetics from a hybrid nanosack according to the disclosure.

FIG. 4 is a graph illustrating glucose-stimulated insulin secretion for 14 days of cultivations (* indicates significant differences in insulin release between low glucose incubation (3 mM) and high glucose incubation (20 mM), p<0.05).

FIG. 5A illustrates the basic structure of an MMP-sensitive, cell adherent peptide having a hydrophobic alkyl tail and its incorporation into the nanofibers sheets according to the disclosure.

FIG. 5B is a series of digital cryo-TEM images showing self-assembly of PA into a nanomatrix gel with calcium ions.

FIG. 5C is a schematic drawing of the encapsulation of islets in the PA nanomatrix gel according to the disclosure.

FIG. 6 schematically illustrates the manufacture and implantation of a hybrid nanosack according to the disclosure.

FIG. 7A illustrates an embodiment of a hybrid nanosack according to the disclosure.

FIG. 7B illustrates an acquired sectioned 2D image of an implanted hybrid nanosack in the omentum of a rat after 2 weeks. Arrows indicates micro-blood vessels invaded inside the hybrid nanosack and purple vessels.

FIG. 7C illustrates an acquired sectioned 3D micro-CT image of an implanted hybrid nanosack in the omentum of a rat after 2 weeks and demonstrates that high-density revasculatures were generated within the hybrid nanosack.

FIG. 8 illustrates the electrospinning of a traditional nanofiber sheet (left) and the electrospinning of an ePCL nanofiber sheet incorporating crater-like structures (right).

FIG. 9 illustrates confocal microscopy images of ePCL nanofiber sheets without (left) and with incorporated crater-like structures (right).

FIG. 10 shows digital images comparing HUVEC infiltration into ePCL nanofiber sheets without (left) and with incorporated crater-like structures (right).

FIG. 11 is a graph illustrating HUVEC proliferation in an ePCL nanofiber sheets with incorporated crater-like structures in response to FGF-1.

FIG. 12 illustrates a modified insert culture system according to the disclosure.

FIG. 13 is a graph illustrating levels of insulin secretion by isolated islets embedded in a nanomatrix according to the disclosure and cultured in a modified insert chamber, non-embedded islets cultured in a modified insert chamber; and isolated islets.

FIG. 14 is a graph illustrating levels of glucose-stimulated insulin secretion normalized to DNA levels by isolated islets embedded in a nanomatrix according to the disclosure and cultured in a modified insert chamber.

FIG. 15 is a graph illustrating levels of glucose-stimulated insulin secretion normalized to DNA levels by non-embedded isolated islets cultured in a modified insert chamber.

FIG. 16 is a graph illustrating levels of glucose-stimulated insulin secretion normalized to DNA levels by islets cultured in tissue culture plates.

FIG. 17 is a series of images illustrating the morphology and viability of rat islets in different culture conditions after 3 days of cultivations. Panel A: bright-field image of islets in the bare group; Panel B: fluorescein diacetate/propidium iodide (FDA/PI) staining of islets in the bare group; Panel C: bright-field image of islets in the insert group; Panel D: FDA/PI staining of islets in the insert group; Panel E: bright-field image of islets in the nanomatrix group; and Panel F: FDA/PI staining of islets in the nanomatrix group. All scale bars indicate 100 μm.

FIG. 18 illustrates the evaluation of insulin-producing β-cells using dithizone staining: Panel A: after 3 days of cultivation in the bare group; Panel B: after 7 days of cultivation in the bare group; Panel C: after 14 days of cultivation in the bare group; Panel D: after 3 days of cultivation in the insert group; Panel E: after 7 days of cultivation in the insert group; Panel F: after 14 days of cultivation in the insert group; Panel G: after 3 days of cultivation in the nanomatrix group; Panel H: after 7 days of cultivation in the nanomatrix group; Panel I: after 14 days of cultivation in the nanomatrix group. All scale bars indicate 100 μm

FIG. 19 illustrates the productivity of insulin, morphology (Bright field images), and viability (FDA/DPI staining) of rat islets in different culture conditions after 7 days of cultivations.

FIG. 20 illustrates the productivity of insulin, morphology (Bright field images), and viability (FDA/DPI staining) of rat islets in different culture conditions after 14 days of cultivations.

FIG. 21A is a graph illustrating non-fasting blood glucose levels for 22 days after transplantation.

FIG. 21B is a graph illustrating intraperitoneal glucose tolerance test at 22 days after transplantation.

FIG. 22 is a series of digital images showing: Panel (a), bright-field image of human islets without nanomatrix gel; Panel (b), bright-field image of human islets with nanomatrix gel after 14 days; Panel (c), FDA/PI staining of human islets without nanomatrix; Panel (d), FDA/PI staining of human islets without nanomatrix gel after 14 days.

FIG. 23 illustrates cross-sectioned images of representational embodiments of collectors: Panel a, 1-3, metal plates; Panel b, 1-3, arrays of metal probes embedded in non-conductive dishes; and Panel c, 1-3, collectors in wet conditions (using organic solvents).

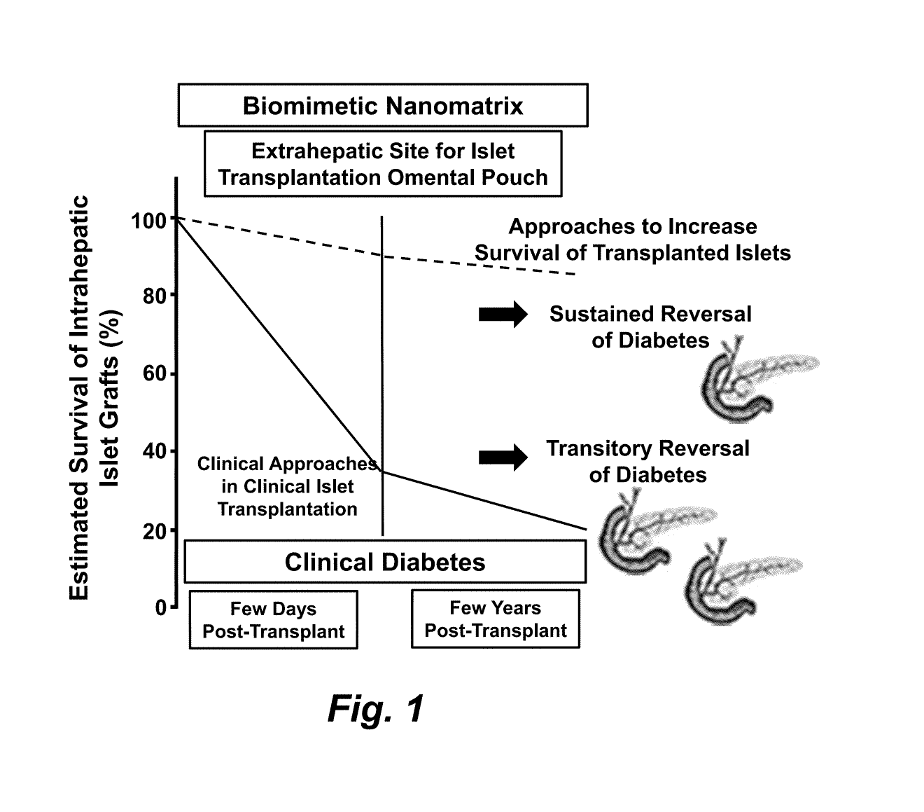

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### DEFINITIONS

The term “peptide amphiphile” as used herein refers to a peptide-based biomaterial that can self-assemble into a nanostructures gel-like scaffold mimicking the chemical and biological complexity of a natural extracellular matrix (see, for example, Lim et al., (2011) Tissue Engineering 17: 399-405, incorporated herein by refrence in its entirety) and having a hydrophobic tail, in particular an alkyl tail. In the peptide amphiphiles of the nanomatrices of the disclosure, it is advantageous for the peptide region to include a cell adherent region such as, but not limited to, the amino acid sequences DGEA, YIGSR, IKVAV, ERGDS, and the like. The peptides may further comprise a region such as metalloproteinase-2 cleavable region. The alkyl tail may be of any suitable composition such as, but not limited to, CH(₂)₂₋₂₀ and advantageously attached to the amino terminus end of the peptide.

The term “cell” as used herein refers to any natural or artificial cell, animal, plant, bacterial, or a viral particle that be viable or dead. Such cells may be isolated from an animal or human subject or tissue thereof, or a cultured cell previously isolated from a subject source. An artificial cell includes, but is not limited to, an artificially engineered entity derived from such as a unicellular microorganism wherein all or some of the genetic material has been replaced.

The term “aggregate” as applied to a “cell” herein refers to a plurality of a cell type or several cell types that may have been dissected (isolated) from a tissue, or have formed a multicellular body upon culturing in vitro. An exemplary aggregate isolated from an animal tissue is a pancreatic islet of Langerhans. Such an islet may include cells other than those identified as β-cells responsive to a stimulus such as glucose and which, in response thereto, synthesize into the surrounding medium insulin. Such an islet may also be formed in such as a liquid medium by culturing isolated pancreatic cells.

The term “biocompatible” as used herein refers to a material that does not elicit any undesirable local or systemic effects in vivo.

The term “scaffold” as used herein refers to a material that can provide a supporting structure on which animal cells may attach and proliferate. The scaffold may be configured to resemble the shape and size of a native animal or human structure or physical feature that it is desired to replace.

The term “extracellular matrix polypeptide” as used herein refers to a polypeptide found in the extracellular matrix of an animal or human tissue including, but not limited to, a fibrous proteins, glycosaminoglycans, heparin sulfate, chondroitin sulfate, keratin sulfate, collagen, elastin, fibronectin, laminin, and the like.

The term “electrospinning” as used herein refers to a process in which fibers are formed from a solution or melt by streaming an electrically charged solution or melt through a hole across a potential gradient. In general, an electrospinning device can include a device (e.g., syringe) and a collection structure. The device is positioned adjacent (e.g., facing the collection structure) collection structure so that fibers can be drawn out of a tip of the device (e.g., tip of the syringe, which is known in the art) or other device across a gap (e.g., a distance of mms to tens of cms) between the device and the collection structure and in the direction of the collection structure based on the potential difference between the tip and the collection structure. Two or more devices can feed fiber to the collection structure from different positions to produce a blend of fibers in the mesh. The fiber can be made of polymers as described herein. For example, but not intended to be limiting, In an embodiment, the fiber can be a nanofiber having a diameter of about 1 to 1000 nm, about 1 nm to 500 nm, about 10 nm to 300 nm, or about 50 nm to 200 nm. An electric field (e.g., about 1 kV/cm to 3 kV/cm) can be produced between the device and the collection structure using appropriate electronic systems. The potential difference between the device and the collection structure (e.g., conductive probes) is about 5 kV to 60 kV or about 20 kV, while the distance between the device and the collection structure is about 5 cm to 30 cm. The potential difference can vary depending on the various distances and dimensions as well as polymers used to make the fiber.

The term “electrospun material” as used herein refers to any molecule or substance that forms a structure or group of structures (such as fibers, webs, or droplet), as a result of the electrospinning process. This material may be natural, synthetic, or a combination of such.

The term “nanofiber sack” as used herein refers to a bag-like structure formed by the folding and sealing by such as tying with a biocompatible fiber and of such size as to enclose a nanomatrix of the disclosure in an amount sufficient for implant into a recipient subject and to, for example, provide a desired level of such as insulin production from islets within the nanomatrix.

The term “nanofiber sheet” as used herein refers to a structure having a third dimension substantially less than that of the other two dimensions. The nanofiber sheets of the disclosure may be electrospun and initially incorporating particles (herein termed “leachable particles”) that once removed from the nanofiber sheet result in the sheet being porous and having surface “crater-like” indentations that have been found to be advantageous for the formation of new vascular structures invading the implants of the disclosure.

The term “leachable particle” as used herein refers to initially solid particles embedded in an electrospun nanofiber sheet during the formation of said sheet. Such particles may be of any material that may be removed from the sheet so as to form a porous sheet or indentations in the surface of the sheet. For example, but not intended to be limiting, the particles may comprise a salt that may be dissolved by a suitable solvent. An especially advantageous leachable particle is a carbonate or a bicarbonate of such as sodium, potassium, ammonium, and the like. When a nanofiber sheet that includes such particles is contacted with an acid, the particles can be converted to soluble salts thereby forming the pores of a porous nanofiber sheet. In addition, the gas, e.g. carbon dioxide generated may contribute both to the formation of the pores and to create the indentations desired to be present on the surface of the nanofiber sheet and which provide a desired advantage of increasing vascular invasion of an implant comprising the nanofiber sheet material used as the exterior surface of a hybrid nanosack implant according to the disclosure.

The term “polymer” as used herein refers to any natural or synthetic molecule that can form long molecular chains, such as polyolefin, polyamides, polyesters, polyurethanes, polypeptides, polysaccharides, and combinations thereof. In particular, the polymer can include: poly (ε-caprolactone), poly vinyl alcohol, polylactic acid (PLA), poly(lactic-co-glycolic) acid (PLGA), poly(etherurethane urea), collagen, elastin, chitosan, or any combination of these.

The terms “growth factor” and “cell growth factor” as used herein refer to molecules that can induce or maintain a cell in a state of proliferation or induce a process such as angiogenesis that is essentially a cell growth process. Suitable growth factors for inclusion in the biomimetic structures of the disclosure are well-known in the art and include, but are not limited to, such as adrenomedullin (AM), angiopoietin (Ang); autocrine motility factor; bone morphogenetic factor (BMPs); brain-derived neurotrophic factor (BDNF); epidermal growth factor (EGF); erythropoietin (EPO); fibroblast growth factor (FGF, FGF-1 and FGF-2); glial cell line-derived neurotrophic factor (GDNF); granulocyte colony-stimulating factor (G-CSF); granulocyte macrophage colony-stimulating factor (GM-CSF); growth differentiation factor-9 (GDF9); hepatocyte growth factor (HGF); hepatoma-derived growth factor (HDGF); insulin-like growth factor (IGF); migration-stimulating factor; myostatin (GDF-8);, nerve growth factor (NGF) and other neurotrophins; platelet-derived growth factor (PDGF); thrombopoietin (TPO); transforming growth factor alpha(TGF-α); transforming growth factor beta (TGF-β); tumor necrosis factor-alpha (TNF-α); vascular endothelial growth factor (VEGF); Wnt Signaling Pathway; placental growth factor (PIGF); (Fetal Bovine Somatotrophin) (FBS); IL-1; IL-2; IL-3; IL-4; IL-5; IL-6.; IL-7, and the like. It is contemplated that more than one type of growth factor may be included in the biomimetic structures of the disclosure. It is further contemplated that the growth factor or combination of growth factors may be embedded within the nanomatrix of the disclosure or attached directly to the polymeric matrix by such as electrostatic or covalent bonds. If other than covalently attached, the growth factor(s) may escape from the nanomatrix gel to enter the surrounding medium or tissues in which it is implanted so as to interact with cells such as vascular endothelial cells, smooth muscle cells, and the like so as to, for example, generate angiogenesis for blood vessel invasion of an implant. It is further contemplated that the growth factor or combination of growth factors may be embedded within or on the surface of the nanofiber sheet of the disclosure or attached directly to the polymeric electrospun fibers thereof by such as electrostatic or covalent bonds. If other than covalently attached, the growth factor(s) may escape from the nanofiber sheet (or nanosack formed from the nanofiber sheet) to enter the surrounding medium or tissues in which it is implanted so as to interact with cells such as vascular endothelial cells, smooth muscle cells, and the like so as to, for example, generate angiogenesis for blood vessel invasion of an implant.

The term “omentum” as used herein may refer to either the grater omentum or the lesser omentum. The greater omentum (also known as the great omentum, omentum majus, gastrocolic omentum, epiploon, or, especially in animals, caul) is a large fold of visceral peritoneum that hangs down from the stomach. It extends from the greater curvature of the stomach, passing in front of the small intestines and reflects on itself to ascend to the transverse colon before reaching to the posterior abdominal wall. The lesser omentum (small omentum; gastrohepatic omentum; Latin: omentum minus) is the double layer of peritoneum that extends from the liver to the lesser curvature of the stomach and the start of the duodenum. It is contemplated, however, that the implantable biomimetic structures of the disclosure may be implanted at any desired site in a recipient animal or human that is compatible to the functioning of the cells embedded in the implants and the desired benefits obtainable from the implants.

### DESCRIPTION

In currently used pancreatic islet implants comprising biomimetic scaffolds, the early loss of β-cell mass and function, and impaired insulin functions followed by β-cell death is attributed to the destruction of the islet extracellular matrix (ECM) microenvironment (Cheng et al., (2011) Tissue engineering. Part B, Reviews 17: 235-247). To improve islet survival and function, studies incorporated isolated ECM proteins into scaffolds. A mixture of different types of collagen increased both rat islet β-cell viability and glucose-stimulated insulin secretion (Nagata et al., (2002) J. Biomater. Sci. Polym. 13: 579-590). Also, human pancreatic β-cells grown on a bovine corneal endothelial cell matrix maintained glucose-stimulated insulin secretion. Collagen IV absorbed in a poly(lactide-co-glycolide) (PLG) scaffold showed enhanced functions of transplanted islets. However, for clinical applications, the use of ECM proteins has some potential problems, such as undesirable immune responses, higher infection risks, variability in biological sources, and increased costs (Hersel et al., (2003) Biomaterials. 24: 4385-4415).

To overcome these limitations, small peptide sequences derived from ECM proteins have been employed to modify the different types of polymers. The growth and function of MIN6 cells were enhanced when encapsulated in photopolymerized PEG hydrogels modified with laminin-derived peptides or type I collagen-derived peptides (Park et al., (2005) J. Biosci. Bioeng. 99: 598-602; Weber et al., (2007) Biomaterials 28: 3004-3011). However, the entrapment of cells in photopolymerized biomaterials can lead to problems after implantation, such as the formation of fibrotic processes, poor degradation of the scaffold, and local and/or systemic toxicity. Differing compositions and concentrations of alginate have also been found to affect the cellular overgrowth of implanted capsules, as metabolic barriers to nutrient diffusion can form around the implant if non-optimal levels of the material are used, despite the established biocompatibility of alginate (King et al., (2001) J. Biomed. Mat. Res. 57: 374-383).

To overcome some of these issues, the disclosure encompasses embodiments of a self-assembled peptide amphiphile (PA) nanomatrix gel that mimics characteristic properties of ECM. The PA nanomatrix gel of the disclosure can enhance the survival and function of encapsulated cells or aggregates of cells such as, but not limited to, pancreatic islets, while providing a protective and nurturing microenvironment.

Peptide amphiphile (PA) nanomatrix gels offer improved efficacy in pancreatic islet engraftment. The use of PA nanomatrix gels as transplant intermediaries for islets, as in the embodiments of the present disclosure, is potentially advantageous because they meet the essential design criteria for synthetically mimicking the ECM: rapid gel-like 3D network formation by self-assembly, versatility to incorporate various cell adhesive moieties, and cell-mediated degradable sites (matrix metalloproteinase-2 (MMP-2)) for progressive scaffold degradation and eventual replacement by host-ECM. Structurally, the PA consists of a hydrophilic functional peptide sequence attached to a hydrophobic alkyl tail, and the internal peptide structure can be adapted to mimic the characteristic properties of the natural ECM. Further, PAs self-assemble into long cylindrical structures that are 8-10 nm in diameter with a length up to several microns in length, and the self-assembly process is initiated by lowering the pH or adding multivalent ions such as, but not limited to, calcium ions, providing biocompatible means to encapsulate islets for engraftment.

It has now been shown that isolated mammalian pancreatic islets can incorporate into the PA nanomatrix gel containing a cell-adhesive ligand isolated from ECM proteins, arginine-glycine-aspartic acid (RGD), as well as an MMP-2 sensitive sequence. The interactions between islets and ECM are known to be important for β-cell viability and function, especially integrin signaling via RGD, which has been shown to decrease apoptosis of islets. Moreover, the MMP-2 enzyme is activated during rat pancreatic development, enabling cell migration of pancreatic endocrine cells throughout the ECM during islet morphogenesis. Thus, the PA nanomatrix gel can facilitate progressive degradation and replacement by host ECM after transplantation in vivo. The PA forms a rapid, viscoelastic 3D microenvironment without any organic solvents or chemicals, providing the islets with a protective and nurturing environment to promote islet survival and function. Hence, PA nanomatrix gels provide an ECM-mimicking microenvironment that imitates the native ECM microenvironment between islets and ECM, thereby improving islet survival and function.

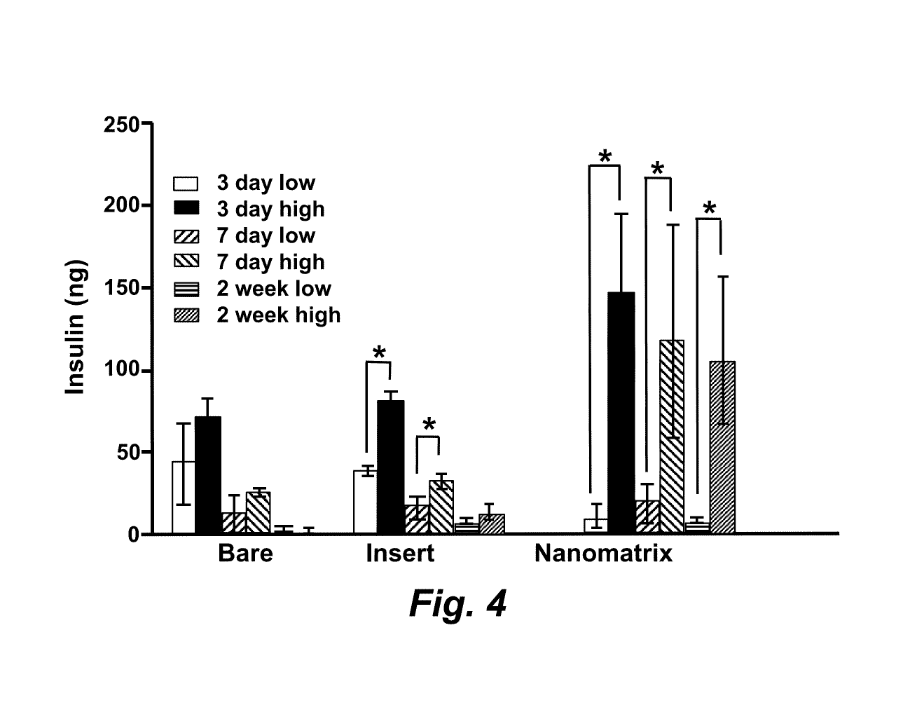

Glucose-stimulated insulin secretion responses were examined in all groups on 3, 7, and 14 days, as shown in FIG. 4. In the nanomatrix group, glucose-induced insulin secretion significantly increased after 7 days and was maintained even after 2 weeks. In the insert group, glucose-induced insulin secretion decreased slightly from 3 days to 7 days and from 7 days to 2 weeks. Even though the total amount of insulin secreted for all three groups at 3 days was the same, both the insert and bare group showed a decrease at 7 days and 2 weeks, while the nanomatrix group showed an increase in secretion. Both in the insert and bare groups, a marked decrease of insulin responses was observed. After two weeks, the bare group showed almost no insulin secretion because most of the islets had lost functionality or had been washed away.

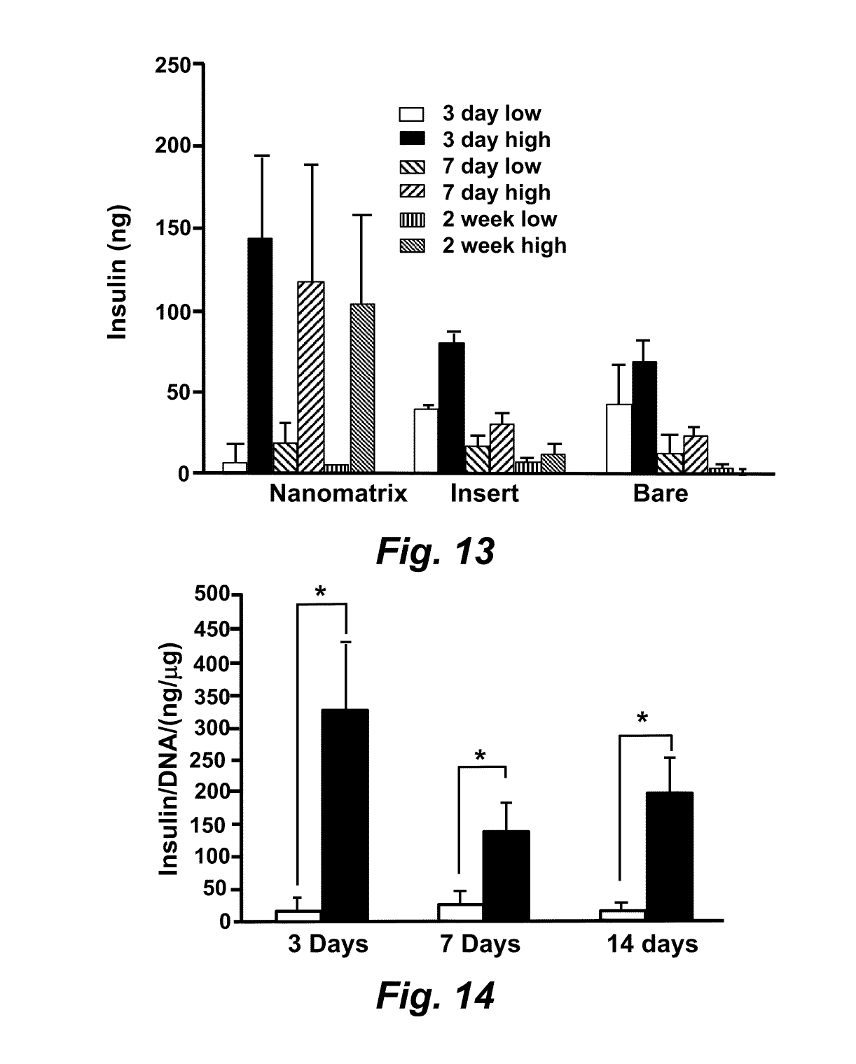

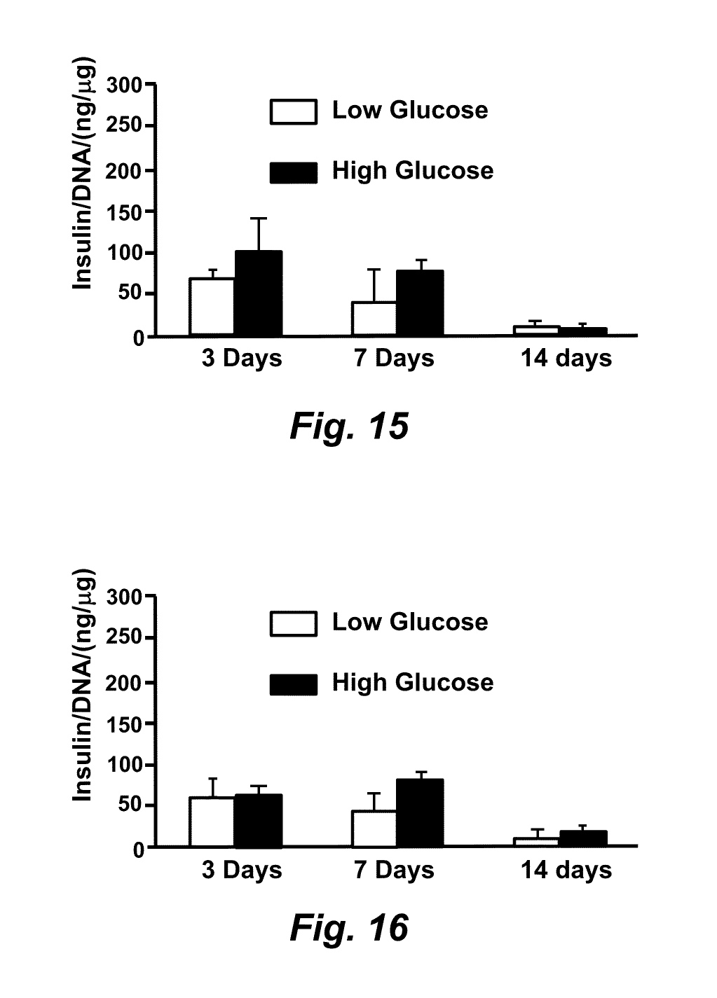

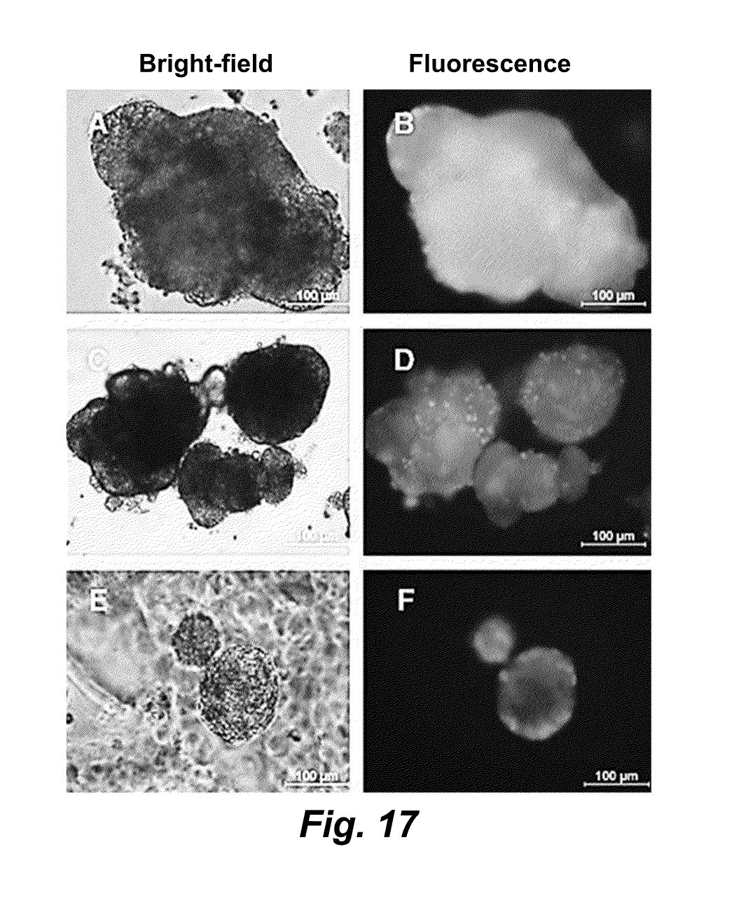

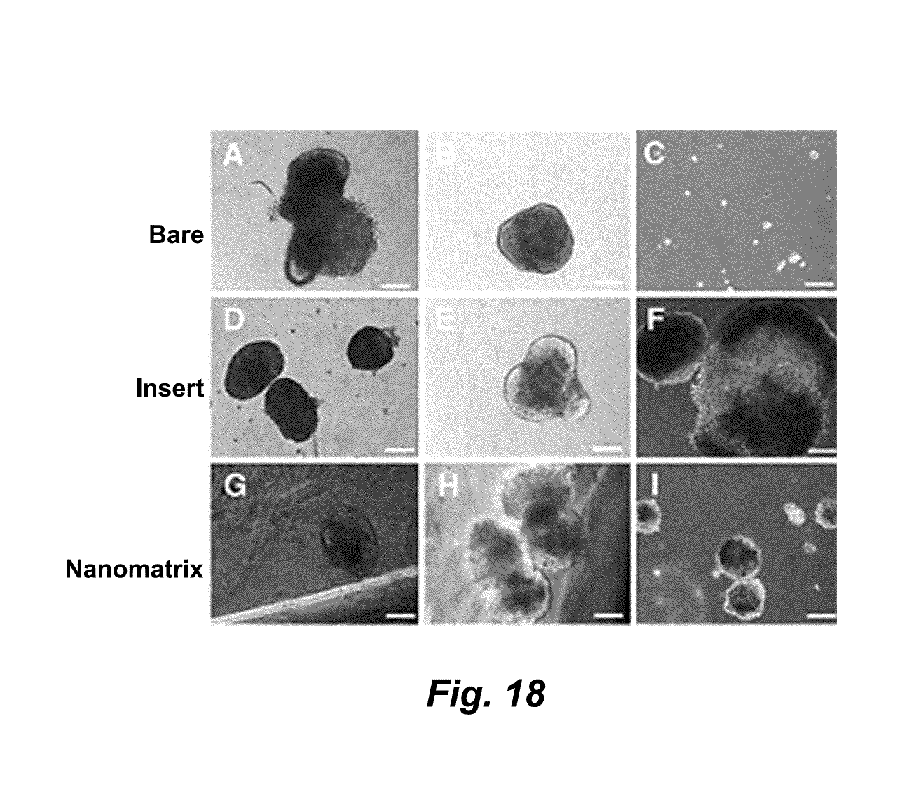

Glucose-stimulated insulin secretion data normalized against DNA showed improved insulin secretion in the nanomatrix group, as shown in FIG. 14. In the nanomatrix group, normalized glucose-induced insulin secretion significantly increased from 3 days to 7 days and from 7 days to 2 weeks. Normalized insulin secretion in the insert and bare groups decreased over time, as shown in FIGS. 15 and 16. Even when comparing the amount of insulin secreted per islet, the secretion for the insert and bare groups was significantly less than that of the nanomatrix group.

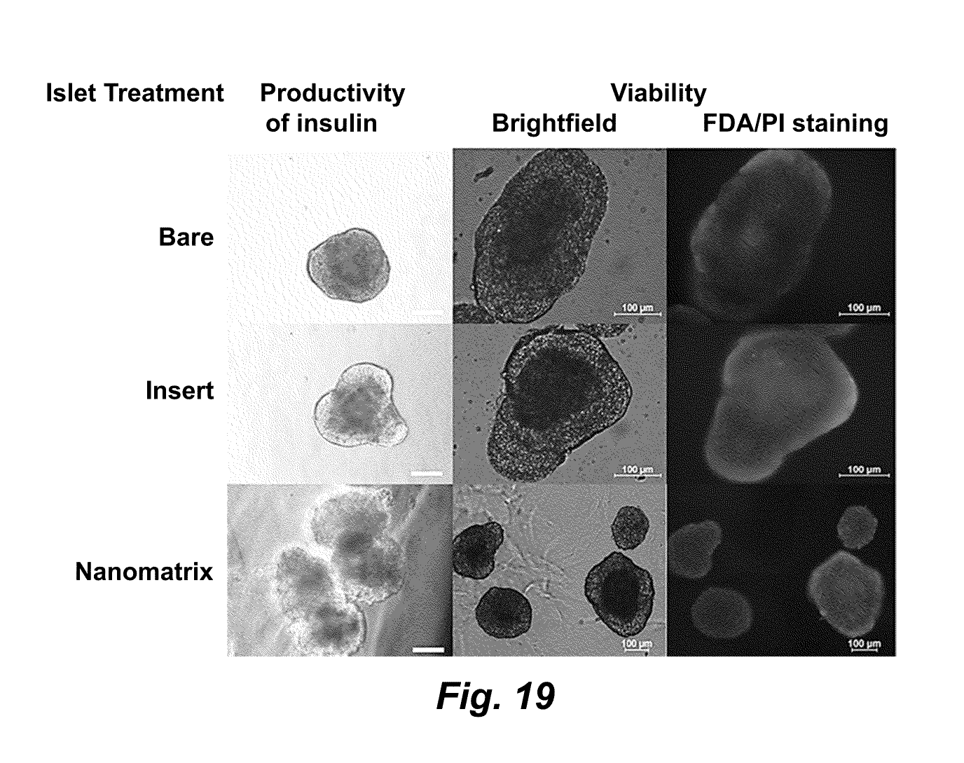

Dithizone staining showed that many β cells in the nanomatrix retained their function even after two weeks, as shown in FIG. 18. A few large-sized islets in the insert group still remained after 2 weeks, but most of islets in the insert and bare group were washed away during cultivation. In the nanomatrix group, even though the islets that remained after 2 weeks were relatively small, they retained most their function.

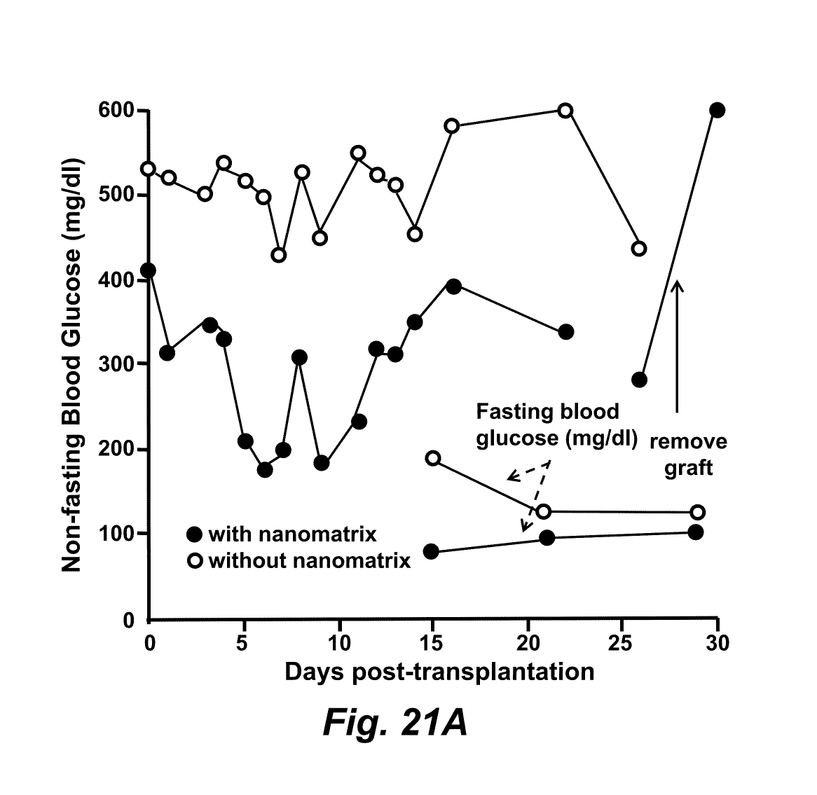

The results of FDA/PI staining showed that the nanomatrix kept individual islets alive even after two weeks of cultivation. In the insert group, relatively large-sized islets remained but were necrotic after two weeks, as shown in FIG. 20. However, the nanomatrix kept the integrity and the viability of the islets.

Thus, biomimetic PAs successfully self-assembled into ECM-mimicking gel like nanomatrix where cell adhesive ligands were inscribed into PAs that encapsulated rat islets.

A modified insert culture system was developed to evaluate islet survival and function, whereby 5 μm nylon meshed inserts were used to hold intact rat islets without physical loss during cultivation. ECM mimicking self-assembled peptide amphiphile (PA) nanomatrix improved islet function and viability. In the PA nanomatrix, rat islets retained their function and enhanced insulin secretion responses to glucose stimulation compared to both bare and insert groups. This nanomatrix also showed the high viability compared to other groups even after 14 days.

The nanomatrix group demonstrated prolonged survival and enhanced function of islets. The glucose-stimulated insulin secretion of nanomatrix group was significantly maintained for up to 14 days, whereas the bare and insert groups showed a much lower level of insulin secretion, as shown in FIG. 4. Additionally, most of the islets were retained in the nanomatrix gel with positive DTZ staining throughout the 14 days of cultivation, demonstrating that the nanomatrix gel provides functional support needed to maintain the oxidative capacity for insulin secretion and granule density for prolonged incubation.

For the insert group, however, fewer islets were observed, which had reduced viability and function, whereas the bare group lost most of its islets over the same period and could not be accurately measured. These findings were consistent with the FDA/PI staining results for all three groups. Thus, throughout the entire cultivation period, the nanomatrix group consistently displayed intact islet integrity with enhanced function, whereas fewer islets remained in the bare and insert groups with reduced utility. Moreover, the nanomatrix gels began to degrade as desired after 14 days due to the inclusion of the MMP-2-sensitive sequence. On the basis of the fact that revascularization begins 2-4 days after islet transplantation and is completed by 10-14 days, these results demonstrate the potential of the nanomatrix gel as a useful intermediary scaffold that bridges the gap between implantation and fully restored host integration.

The normalization of islet quantification data is important for accurately reflecting the overall islet performance. However, the traditional normalization methods do not account for variations in the viability and number of islets, which can both be altered due to the physical loss that occurs during the cultivation. Thus, traditional methods frequently lead to misinterpretation of the data, especially in long-term studies. Consequently, the glucose-stimulated insulin secretion values were normalized by the amount of DNA per sample to reduce variations and account for the different numbers of remaining islets in each condition. It was found that not only were islets in the nanomatrix group producing significantly more total insulin, but also, when normalized by total islet DNA, the islets in the nanomatrix group showed more insulin per DNA than the bare and insert groups, as shown in FIGS. 14-16. 7. In contrast, there were marked decreases in the bare and insert groups when normalized by DNA. β-cell proliferation during cultivation affected the normalized islet function data, as cultured β-cells very rarely proliferate under normal culture conditions (Ouziel-Yahalom et al., (2006) Biochem. Biophys. Res. Commun. 341:291; Weinberg et al., (2007) Diabetes 56: 1299). Thus, these results indicate that not only were more islets retained in the nanomatrix group, but also the individually remaining islets demonstrated enhanced survival and function per DNA.

The data of the disclosure indicate that the ECM-mimicking PA nanomatrix gel can provide a protective and nurturing microenvironment that enhances islet cell survival, and, most importantly, increases function in the β-cell mass in vivo. Accordingly, peptide amphiphile (PA) is a peptide-based biomaterial that can self-assemble into a nanostructured gel-like scaffold, mimicking the chemical and biological complexity of natural extracellular matrix.

To evaluate the capacity of the PA scaffold to improve islet function and survival in vitro, rat islets were cultured in three different groups: (i) bare group: isolated rat islets cultured in a 12-well non-tissue culture-treated plate; (ii) insert group: isolated rat islets cultured in modified insert chambers; and (iii) nanomatrix group: isolated rat islets encapsulated within the PA nanomatrix gel and cultured in modified insert chambers, and shown in Table 1

TABLE 1

| | |
|---|---|
| Nanomatrix | Encapsulated with nanomatrix and cultured in the modified |
| | insert culture system |
| Insert | Cultured in the modified insert system without nanomatrix |
| Base | Cultured directly in a 48-well, non-tissue culture-treated |
| | plate |

Over 14 days, both the bare and insert groups showed a marked decrease in insulin secretion, whereas the nanomatrix group maintained glucose-stimulated insulin secretion. Moreover, entire islets in the nanomatrix gel stained positive for dithizone up to 14 days, indicating better maintained glucose-stimulated insulin production. Fluorescein diacetate/propidium iodide staining results also verified necrosis in the bare and insert groups after 7 days, whereas the PA nanomatrix gel maintained islet viability after 14 days. These results demonstrated the potential of PAs as an intermediary scaffold for increasing the efficacy of pancreatic islet transplantation.

### Electrospinning:

Traditional electrospinning produces flat, highly interconnected scaffolds consisting of densely packed nanofibers. These electrospun scaffolds can support the adhesion, growth, and function of various cell types, while also promoting their maturation into specific tissue lineages, such as bone, cartilage, tendons, ligaments, skin, neurons, liver, smooth muscle, striated muscle, and even cornea. In addition, the morphology of electrospun nanofibrous scaffolds is highly tunable by simply modifying any number of fabrication parameters, such as concentration of polymer solution or voltage between nozzle and collector such as described by Pham et al., (2006) Tissue Eng. 12: 1197-211). This is advantageous for tissue engineering systems because it has been shown that the fiber diameter, pore size, and even solvent used affect cellular response to electrospun biomaterials. However, a major limitation of traditional electrospun scaffolds is that they have tightly packed layers of nanofibers with only a superficially porous network, resulting in confinement to sheet-like formations only. This unavoidable characteristic restricts cell infiltration and growth through the scaffolds. Thus, it was a need to develop methods of fabricating an electrospun scaffold with a stable three dimensional structure, while exhibiting nanofibrous morphologies and deep, interconnected pores. Such a scaffold can better mimic the configuration of native extracellular matrix (ECM), thereby maximizing the likelihood of long-term cell survival and generation of functional tissue within a biomimetic environment.

While providing biocompatibility, biodegradability, and unique mechanical properties, tissue engineering scaffolds should require a certain degree of porosity and interconnectivity for cell infiltration and tissue growth. Importantly, it has been challenging for electrospun nanofibers to create highly porous structures for blood vessel infiltration. Thus, highly porous crater-like structures have been successfully achieved using the methods of the disclosure that incorporate salt-leaching techniques to develop a timely-sprinkled particulate method, in which a combination of sprinkling a variety of organic particles with varying diameters with a predetermined interval of times are used to obtain a crater-like structured nanofiber sheet made by electrospinning the poly (ε-caprolactone) polymer. This has allowed the fabrication of such as a poly (ε-caprolactone) electrospun (ePCL) nanofiber sheet with crater-like porous structures, enabling high porosity with a nominal diameter of approximately 500 nm, and a random, interwoven network arrangement.

The techniques used for traditional electrospinning employ a static, flat-plate collector placed at a set distance away from a charged nozzle containing a polymer solution. The resulting electrospun scaffolds are composed of nanofibrous layers arranged in a tightly packed conformation, which allows cellular growth and infiltration near the superficial surface but not deep within the internal structure. Many potential solutions have been investigated to improve this scaffold deficiency; however, the paradoxical nature of the electrospinning process works against achieving an ideal formation that allows for both good cell attachment and deep cellular infiltration. Specifically, as the fiber diameter decreases to the nanoscale range for optimal cell attachment, the porosity decreases as well, thereby preventing deep cellular infiltration that is most easily overcome by reverting back to microscaled fiber diameters (Eichhorn & Sampson (2005) J. R. Soc. Interface 2: 309-318). This drawback has previously discouraged exclusively electrospun scaffolds, and has led to exploration of other electrospun nanofiber uses, such as coatings for more porous scaffold material including microfibers.

A particularly useful method for the generation of electrospun materials for use in the nanosacks of the disclosure utilizes salts dissolved in the polymer solution to create specific pore sizes throughout the scaffold by leaching out the particulates after electrospinning, as described by Kim et al., (2008) Acta Biomaterialia. 4: 1611-1619 and Nam et al., (2007) Tissue Eng. 13: 2249-2257. This forms porous spaces in the scaffold; however, the spaces act as a divider for creating separate layers within the scaffold, much like layering multiple scaffolds, which does not provide uniform morphology and stability.

The basic method to electrospin polymer fibers is to place a grounded collector near a charged syringe nozzle, which contains a conductive polymer solution. As the applied voltage is increased, the solution overcomes the frictional forces, resulting in a spinning jet of polymer fluid being ejected from the needle. This ejected solution evaporates as it travels over the projected distance, depositing a mesh of fibers on the collector. The resulting fiber characteristics are largely determined by the solution viscosity, flow rate, and distance between nozzle and collector. Low viscosities, low flow rates, and large distances generally result in smaller diameters. However, the overall scaffold characteristics are largely determined by the collector.

On a traditional flat-plate collector, the grounded charge is spread uniformly over a large area. As a result, a group of fibers is deposited side-by-side in one layer, and each subsequent layer is deposited on top of the existing layers. However, each layer is still strongly attracted to the grounded collector, thus compressing the layers below. This creates a flat, sheet-like structure with densely packed fiber layers and superficial, planar pores, which do not continue deep into the scaffold. While the accumulated fiber layers do provide a thickness to the scaffold, the lack of space between adjacent layers essentially creates a two dimensional scaffold, especially since cellular growth and infiltration are limited to the superficial layers.

Therefore, to create an electrospun scaffold with nanofibrous morphologies and deep, interconnected pores incorporated within a more realized three dimensional structure, the traditional collector was replaced with a non-conductive spherical dish that has an array of embedded metal probes. This arrangement evenly dispersed and concentrated the grounded charge on the probes. The probes were then able to collect the nanofibers between them in mid-air, and the lack of a uniform charge throughout the collector allowed nanofiber layers to settle next to the previously deposited layers without compressing the scaffold. In addition, the spherical dish helped collect the nanofibers in a focused area, thereby accumulating them as a fluffy, three-dimensional structure with good stability.

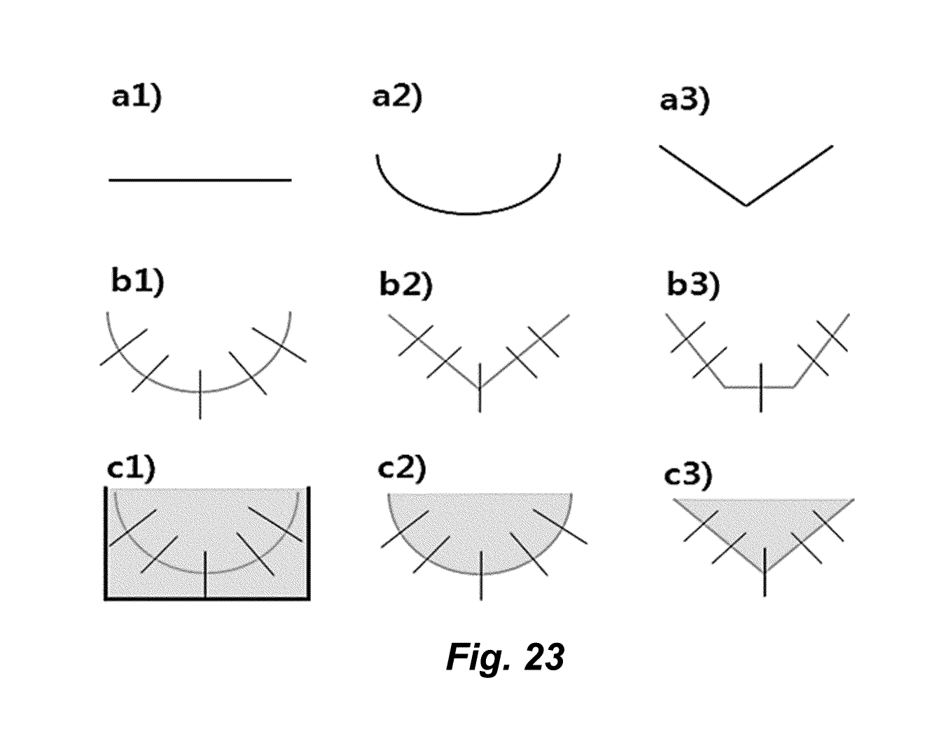

The collector system has a dramatic influence on overall scaffold characteristics. As a result of the uniformly concentrated charge of the traditional collector, the generated scaffold has a very tightly packed structure assembled as in a flat, sheet-like arrangement. In contrast, the spherical dish and metal array collector herein disclosed created a focused, low density, and uncompressed nanofibrous mesh with significant three dimensional depth. Thus, the collector provides an alternative strategy for overcoming one of the current challenges facing electrospinning fabrication, as new scaffolds were created with a stable and interconnected nanofibrous architecture in multiple planes. However, while not intending to be limiting, embodiments of the collector suitable for use in the electrospinning of the nanofiber sheets according to the disclosure, are shown, for example, in FIG. 23.

A variety of polymers can be modified to obtain functional properties and design flexibility desirous in a scaffold. Similarly, biodegradability can be achieved by tailoring some of these polymers (Murugan & Ramakrishna (2007) Tissue Eng. 13: 1845-1866). As such, embodiments of biomimetic implantable scaffolds of the disclosure may advantageously comprise such as poly(lactic acid), poly(caprolactone), or a combination thereof.

Poly(lactic acid) (PLA) as used herein refers to an aliphatic polyester derived from renewable resources, such as corn starch or sugarcane. It is a biodegradable thermoplastic, and the degradation product lactic acid is metabolically innocuous, making it an advantageous material for medical applications. As such, it is one of the few biodegradable polymers approved for human clinical use.

The degradation of PLA involves random hydrolysis of its ester bonds to form lactic acid that enters the tricarboxylic acid cycle to be excreted as water and carbon dioxide. The degradation rate can vary by altering factors such as structural configuration, morphology, stresses, crystallinity, molecular weight, copolymer ratio, amount of residual monomer, porosity and site of implantation, and the like, by methods well known to those in the art.

For the generation of the nanofibers of the nanofiber sheet of the disclosure, poly (ε-caprolactone) (PCL) was selected as an especially advantageous polymer because it is biocompatible and approved for use in biomedical applications. Polycaprolactone (PCL) is derived by chemical synthesis from petroleum. It is a semi-crystalline, resorbable, aliphatic polyester that biodegrades by hydrolysis of ester linkages and eventual intracellular phagocytosis. PCL degrades at a lower rate than PLA and is useful in long term, implantable drug delivery systems.

Furthermore, PCL can be readily electrospun into nanofibers (ePCL). The biological response of the ePCL electrospun scaffolds with a rat insulinoma INS-1 (832/13) cells (INS-1 cells) cell line was examined. INS-1 cells are a very robust cell line that allow for quick and easily obtained biological analysis. Furthermore, this cell line was developed to mimic β-cell function (Asfari et al., (1992) Endocrinology 130: 167-178; Hohmeier et al., (2000) Diabetes 49: 424-430; Yang et al., (2004) Mol. Endocrinol. 18: 2312-2320), which has great utility for studying pancreatic tissue engineering applications.

ECM functionality is highly regulated by complex cellular interactions with different fibrillar proteins that perform biological activities at the nanoscale dimension (Daley et al., (2008) J. Cell Sci. 121: 255-264; Hubbell J A. (2003) Curr. Opin. Biotechnol. 14: 551-558; Kleinman et al., (2003) Curr. Opin. Biotechnol. 14: 526-532; Streuli C. (1999) Curr. Opin. Cell Biol. 11:643-640). Numerous reports have also demonstrated a positive influence of nanofibrous biomaterial structures on cellular activity (Li et al., (2006) Tissue Eng. 17: 1775-1785; Kwon et al., (2005) Biomaterials 26: 3929-3939). Hence, the scaffold parameters designed for this study were specifically chosen to create electrospun nanofibers that were similar in scale to native ECM macromolecules. The majority of fiber diameters in the traditional ePCL scaffolds were between 300-400 nm, while the ePCL scaffolds of the disclosure displayed fiber morphologies with an approximate diameter of 500 nm, both within the typical size range of collagen fiber bundles found in native ECM. Additionally, even with the different parameters (PCL concentration, flow rate, and voltage), the 2D and 3D nanofiber characteristics were similar. However, the overall scaffold morphologies were significantly affected by the collectors: the traditional collector generated a tightly packed fibrous network while the embodiments of the collector, as shown, for example in FIG. 23, are able to create an uncompressed, loosely packed, and more porous nanofibrous structure.

The Omentum as an Islet Transplantation Site with Enhanced Revascularization to Overcome Limitations of Current Intrahepatic Implantation Sites:

While the portal vein has been widely used for intrahepatic PIT, it is associated with procedural risk, islet damage by “instant blood mediated inflammatory reaction” (IBMIR), progressive attrition of islet function, exposure to the toxic effects of immunosuppressive drugs, exposure to toxic products from the gastrointestinal tract, etc. (Barshes et al., (2005) Leukoc. Biol. 77: 587-597; Contreras J L. (2008) Xenotransplantation 2: 99-101; Korsgren (2008) Diabetologia 51:227-232; Robertson R P. (2002) J. Clin. Endocrinol. Metab. 87: 5416-5417; Windt et al., (2007) Xenotransplantation 14: 288-297). The omentum, however, can be engineered for islet implantation with immunological privilege and a large implantation volume. Although the natural properties of the omentum have demonstrated beneficial effects in the islet transplant setting, technical problems related to the “leakage” of islets from the omentum and higher numbers of islets required to establish euglycemia have been identified. In particular, islet revascularization is a crucial factor in improving the survival and function of islet grafts in the omentum. The hybrid nanosack of the disclosure has unique properties for transplantation of cells or a cell aggregates such as islets, including the controlled release of vascularization-stimulating growth factors such as, but not limited to, FGF-1, FGF-2, VEGF, and the like and well-known to those in the art, suitable mechanical properties for surgical manipulation, and enhanced revascularization in the omentum.

The hybrid nanosacks of the disclosure provide an islet ECM-mimicking microenvironment to enhance islet survival, function, and engraftment in the omentum. The hybrid nanosack has been designed for both mimicking an islet ECM microenvironment and inducing rapid revasculazation in the omentum. The embodiments of the hybrid nanosack of the disclosure, therefore, include a self-assembled peptide amphiphile (PA) nanomatrix gel capable of encapsulating islets with a nurturing microenvironment, and an electrospun nanofiber sheet with crater-like porous structures advantageous for the infiltration of blood vessels, while further providing a mechanically stable structure for surgical manipulation.

Rapid gel formation by self-assembly at physiological conditions, the versatility to incorporate cell adhesive ligands, enzyme-mediated degradation (by such as matrix metalloproteinase-2, MMP-2), and the ability to release growth factors in a highly controlled manner are advantages offered by the nanosacks of the disclosure. In particular, it has been found that the PA-RGDS nanomatrix gels of the disclosure show substantial enhanced islet function and viability when compared to current methods of preparing islets for implantation into a subject in need of.

The hybrid nanosacks of the disclosure allow a sustained growth factor (such as but not limited to, fibroblast growth factor-1 (FGF-1) or fibroblast growth factor-2 (FGF-2)) release in a multi-stage release kinetic for enhanced revascularization in the omentum. Although the use of FGF-1 and FGF-2 have been shown, it is contemplated that any suitable growth factor may be included in the matrices of the disclosure for the promotion of implanted cell growth or the vascularization of the implants.

For example, but not intended to be limiting, an initial burst release of FGF-2 from the electrospun nanofiber sheet to stimulate the angiogenic process can be followed by a sustained release of FGF-2 from the PA nanomatrix gel to promote the enhanced islet engraftment is contemplated. Rapid revascularization between the transplanted islets and the systemic circulation is important for successful long-term islet engraftment. The death of islets is most likely associated with ischemia and inadequate blood supply derived from an incomplete revascularization, and the extent of the revascularization depends on the anatomical site of implantations.

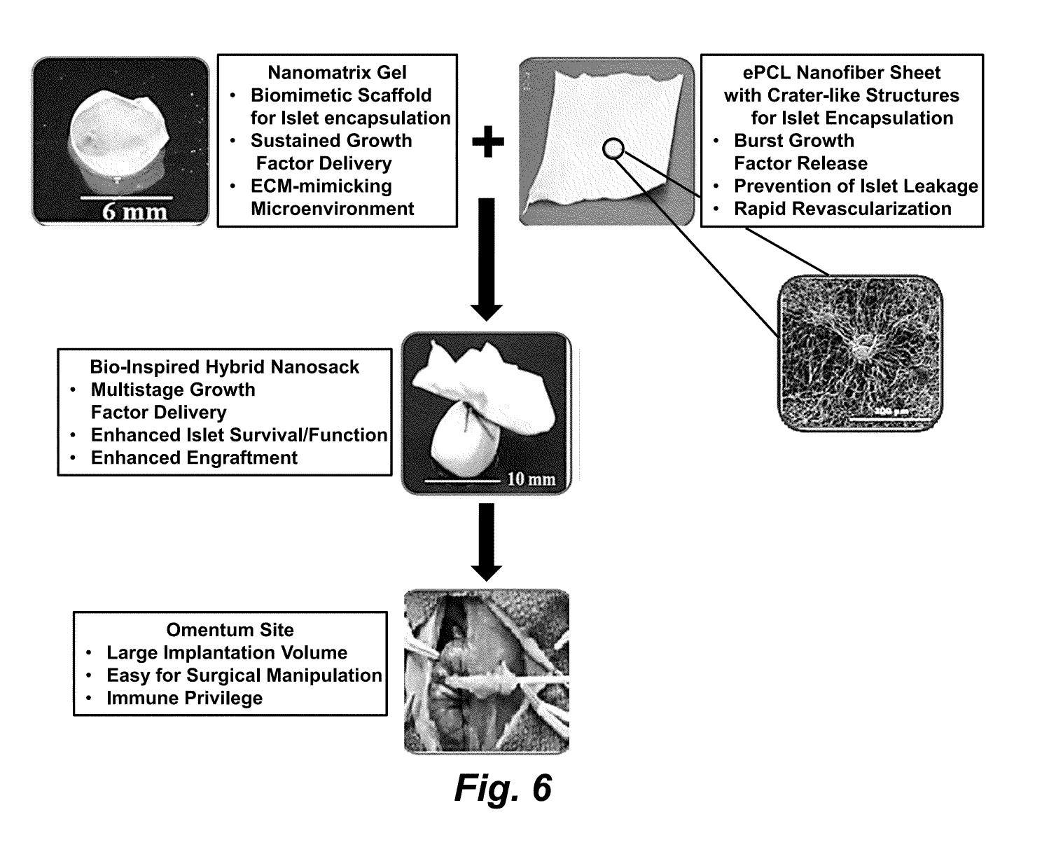

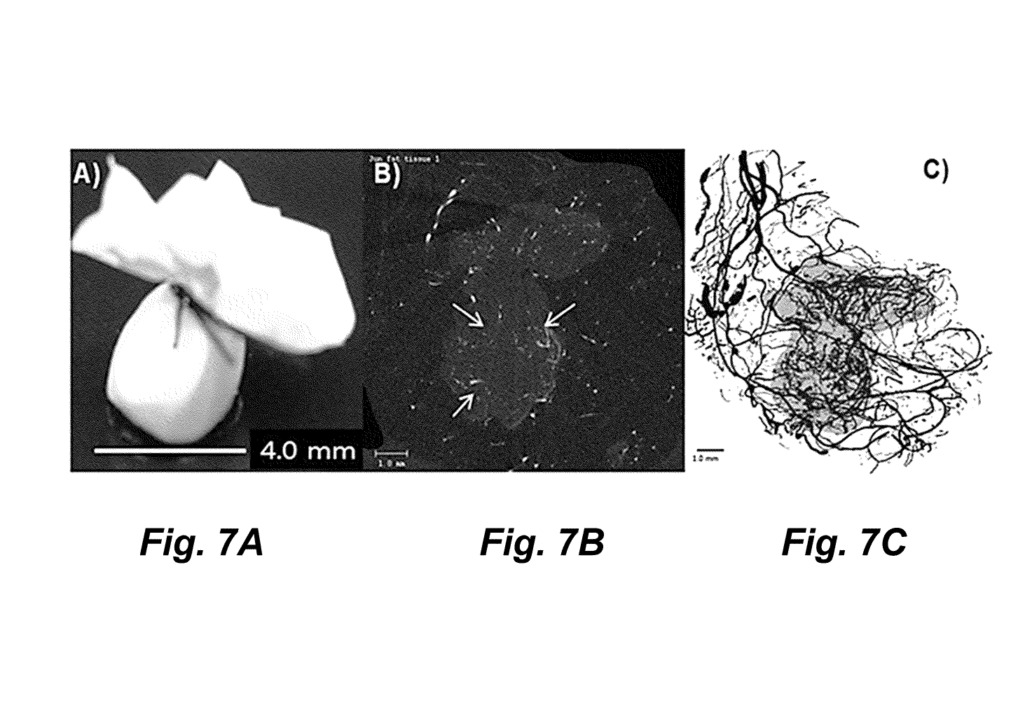

It has further been found that the inclusion of novel porous crater-like structures of the nanofiber sheets of the disclosure facilitate the infiltration of blood vessels and promote the rapid vascularization of the implants in the omentum as shown, for example, in FIGS. 7A-7C.

### Highly Porous Crater-Like Structures:

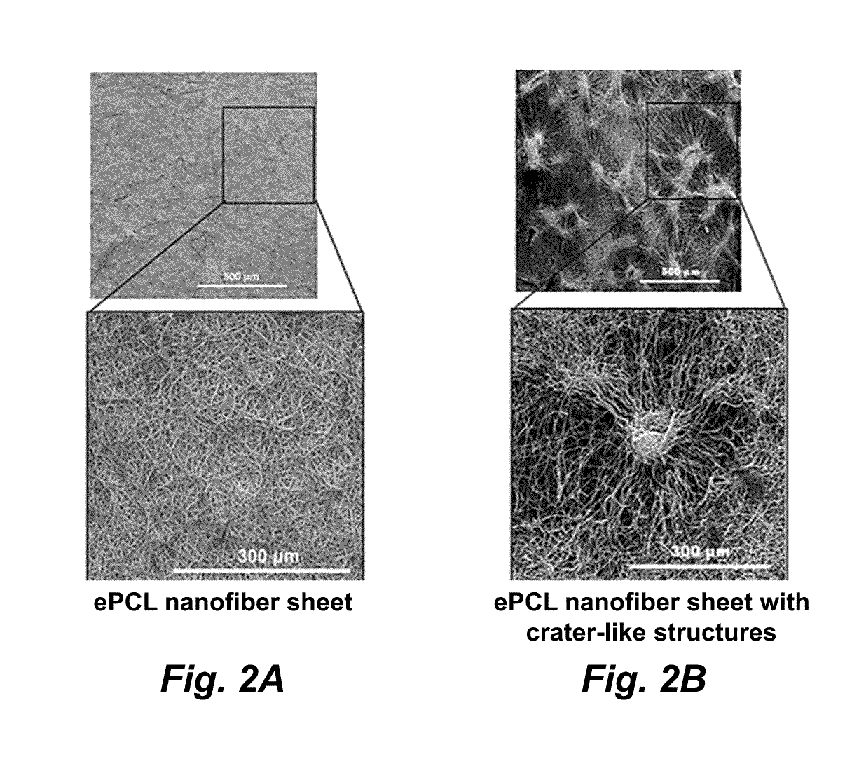

The ePCL nanofibers of the disclosure have been fabricated with a diameter of approximately 500 nm, and exhibit a random, interwoven network arrangement as described previously (Andukuri et al., (2011) Acta Biomaterialia 7: 225-233; Tambralli et al., (2009) Biofabrication. 1: 025001; Moya et al., (2010) J. Surg. Res. 160: 208-212). The highly porous crater-like structures have been successfully achieved using the methods of the disclosure, as demonstrated in SEM images in FIGS. 2A and 2B.

To satisfy the need for an increase in the efficacy of pancreatic islet transplantation at the omentum site, embodiments of a bio-inspired hybrid nanosack were developed that combined a peptide amphiphile (PA) nanomatrix gel with an electrospun biodegradable poly caprolactone (e-PCL) nanofiber sheet that includes crater like structures. The hybrid nanosack was designed to have the synergistic characteristics of two materials: a) the encapsulation of islets within an extracellular matrix mimicking environment, and b) angiogenic factors such as, but not limited to, a fibroblast growth factor (e.g. FGF-1) that may be released in a controlled manner and incorporated into a mechanically stable protective structure for surgical manipulation.

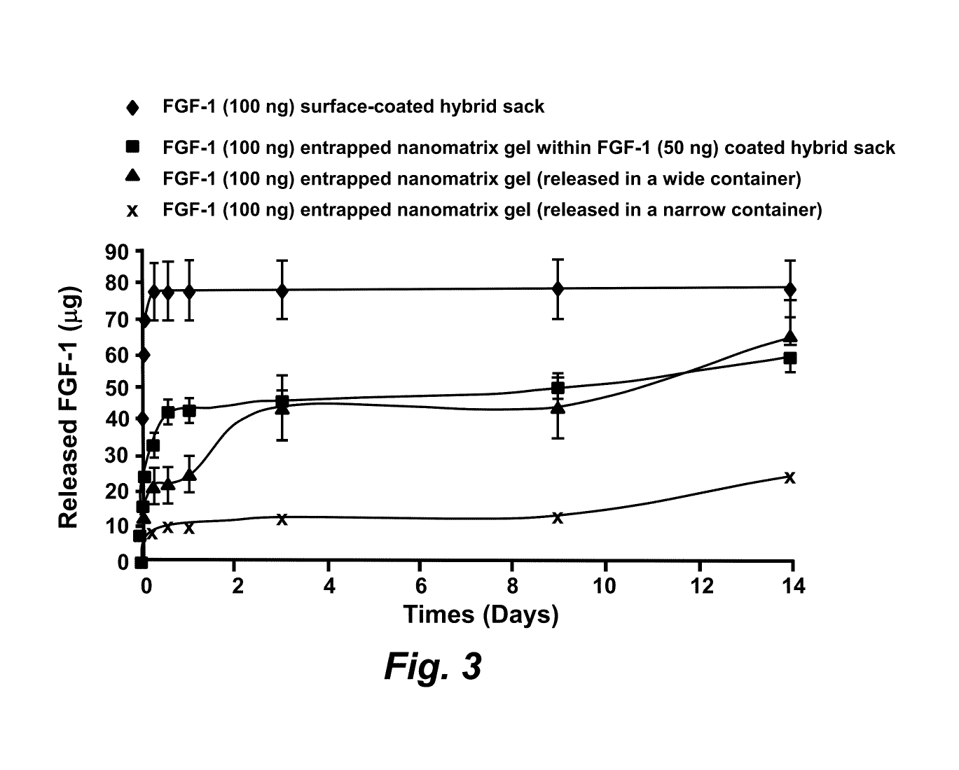

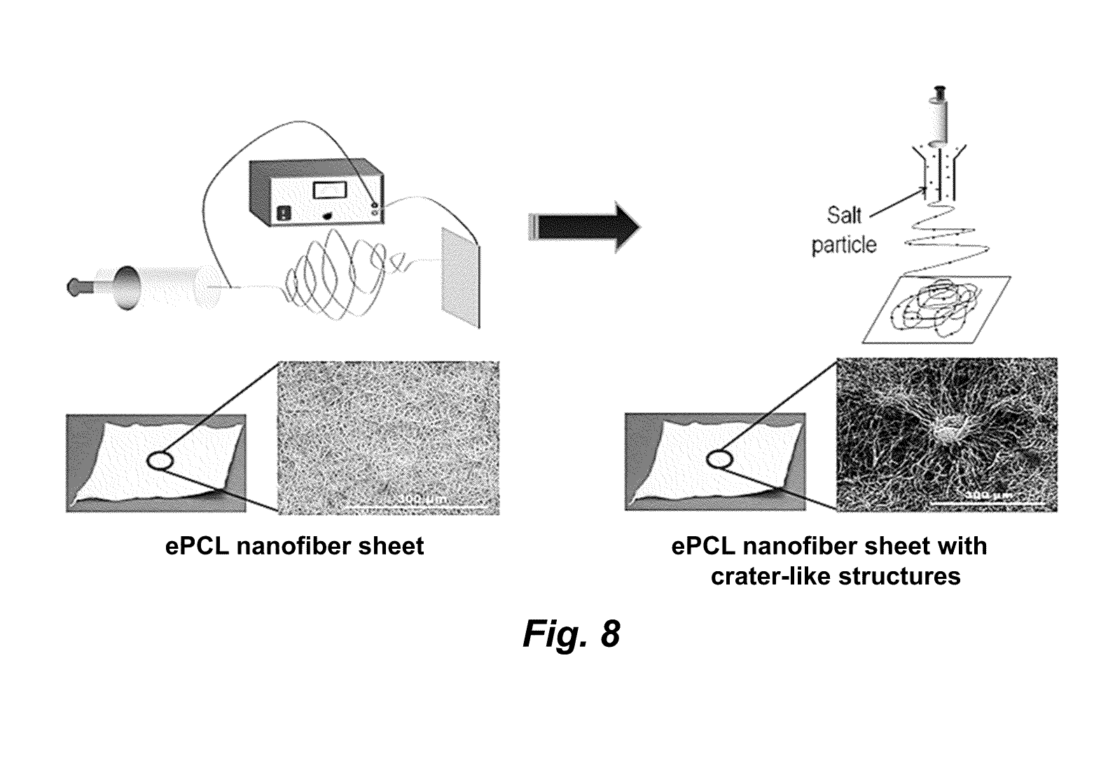

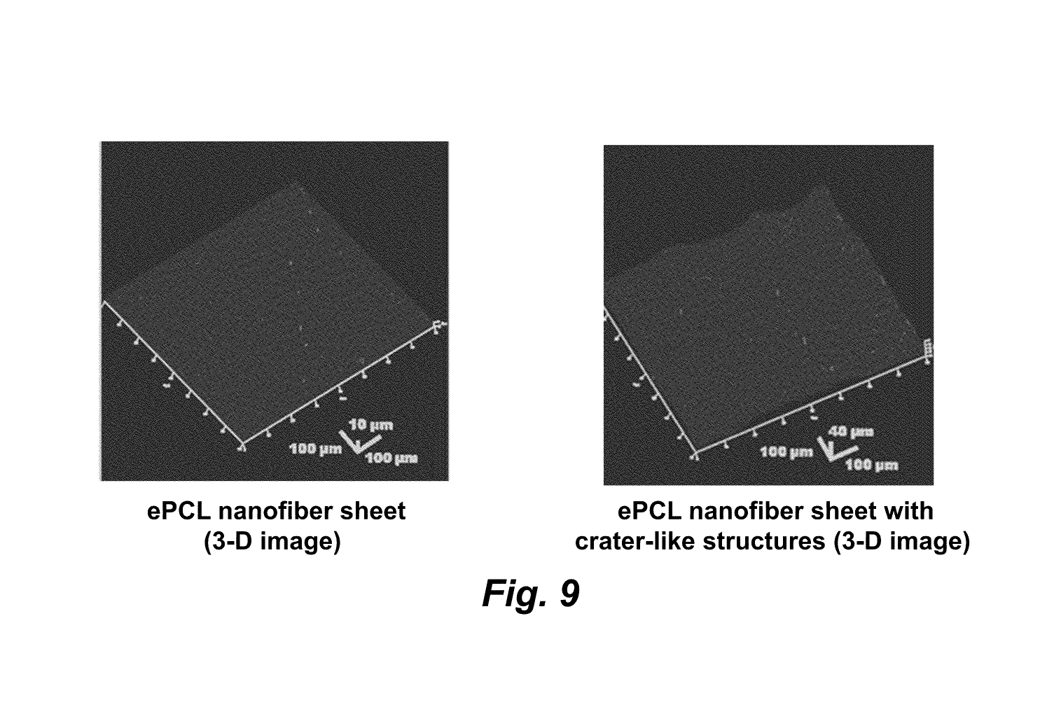

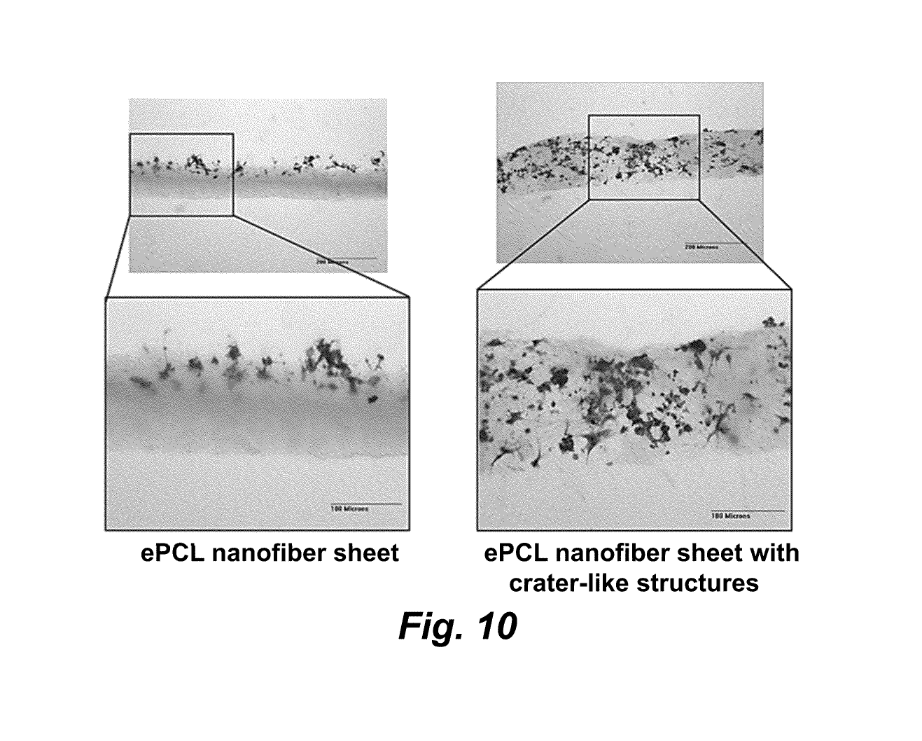

Accordingly, embodiments of the implantable sacs of the disclosure were generated by forming crater like structures in ePCL nanofiber sheet that were fabricated by a gas foaming/salt leaching technique and characterized by scanning electron microscope (SEM) and 3-D confocal microscopy, as shown, for example, in FIGS. 2 and 9. This crater like structure allows newly-generated blood vessels to penetrate more easily through PCL sheet. In addition, the incorporation of growth factors that stimulate vascularization of the sacs of the disclosure has been demonstrated. Thus, FGF-1 bioactivity (picogreen assay), and the release kinetics of FGF-1 from hybrid sack (ELISA assay) have been studied. FGF-1 stimulated human umbilical vein endothelial cells (HUVEC) proliferation and the hybrid sacks of the disclosure showed multi-stage FGF-1 release kinetics, as shown in FIG. 3, and this can enhance angiogenesis at an omentum site of implantation.

Pancreatic islet transplantation (PIT) has been given increasing attention as an alternative treatment for insulin-dependent diabetes mellitus, but a few limitations to its success in clinical trials have been identified. In particular, the substantial loss of islets is reported as one of primary causes of islet graft failure because the destruction of extracellular matrix (ECM) around islets causes reduced β-cell function and survival. To address this issue, the present disclosure provides embodiments of a self-assembled peptide amphiphile (PA) nanomatrix gel suitable for providing a protective and nurturing ECM microenvironment for the survival and functioning of isolated rat islets.

Accordingly, dissected isolated rat islets were cultured over a 14 day period either within or without the self-assembled PA nanomatrix gels of the disclosure. Glucose-stimulated insulin secretion was measured for 14 days. Islet viability was assessed with a fluorescein diacetate/propidium iodide (FDA/PI) staining, and insulin production in islets was assessed with a dithizone (DTZ) staining. Additionally, 1500 syngeneic rat islets were encapsulated within the nanomatrix gel and transplanted into the left renal subcapsule of STZ-induced diabetic rats to evaluate the efficacy of PA nanomatrix gel in vivo.

For bare isolated islets without the nanomatrix gel, there was a marked decrease in glucose-stimulated insulin secretion, whereas islets encapsulated within the nanomatrix gel showed maintenance of function, even over 14 days, as shown in FIG. 4. There was also a trend towards a significant reduction in blood glucose levels within the implanted nanomatrix gel group of animals. Thus the PA nanomatrix gel is useful as an intermediary scaffold for increasing the efficacy of pancreatic islet transplantation.

Pancreatic islet transplantation (PIT) has demonstrated consistent and sustained reversal of type 1 diabetes, generating optimism for wider application of PIT as a potential cure for type 1 diabetes. The omentum site is an attractive transplantation site for PIT as it can accommodate larger implantation volumes, the concurrent use of transplant devices, and some immune privilege (Table 2).

TABLE 2
Advantages and disadvantages of alternative pancreatic
islet transplantation sites.

| Optimal Site | ^{a}Liver | Omentum |
|---|---|---|
| Local immune protection | No | Yes |
| Drainage directly to the liver | Yes | Yes |
| Temperature of body core | No | Yes |
| Biopsis sampling | No | Yes |
| Minimal invasive surgery | Yes | No |

^{a}Immediate destruction of 50-60% transplanted cells

However, vascularization of the implanted material is one of the major challenges of the omentum site due to its low vascularity.

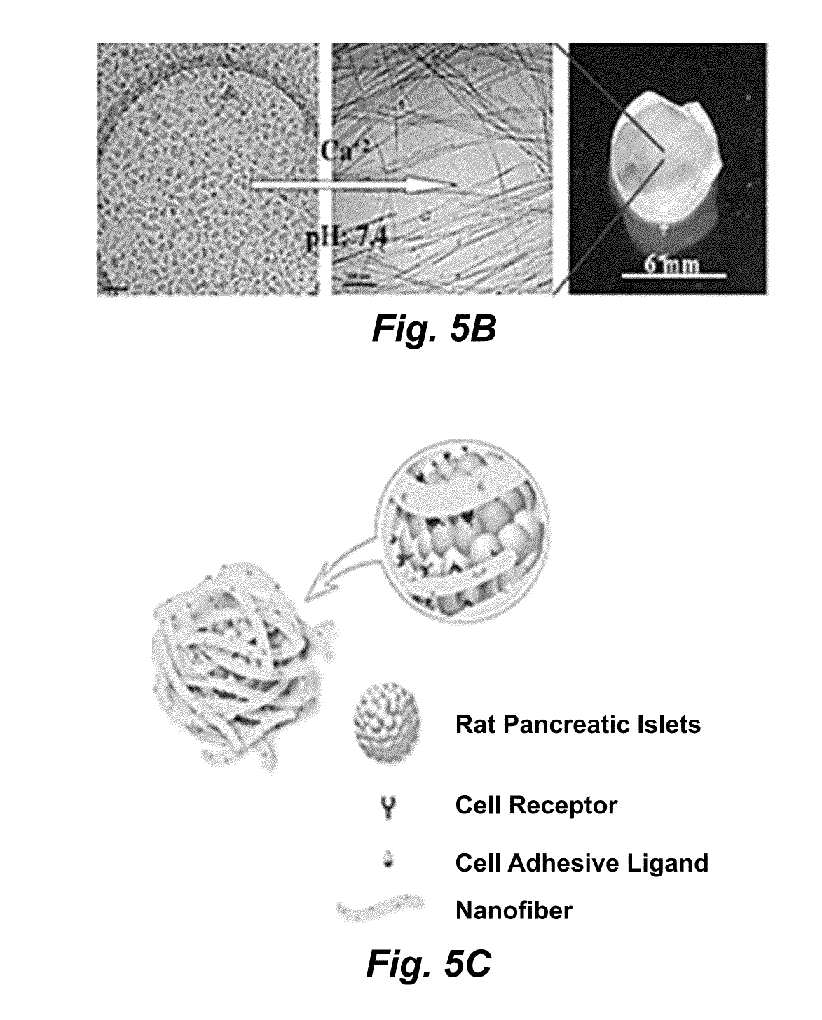

To stimulate vascularization of the implants at the omentum site, the controlled delivery of a fibroblast growth factor to the omentum site would be advantageous. For example, but not intended to be limiting, an initial burst release of FGF could stimulate de novo angiogenesis. A subsequent more sustained release of FGF would allow the transplanted islets to be surrounded with a stable vascular network. Accordingly, embodiments of a biocompatible biomimetic inspired hybrid nanosack was developed to increase the efficacy of PIT at the omentum site. The hybrid nanosack provided by the disclosure combines a self-assembled peptide amphiphile (PA) nanomatrix gel and an electrospun polycaprolactone (ePCL) nanofiber sheet with crater like structures, which provides multistage kinetics of FGF for revascularization, an encapsulation of islets with an extracellular matrix mimicking environment, and a mechanically stable protective structure for surgical manipulation at the omentum site. A scheme for the manufacture of the bio-compatible sacks of the disclosure is shown in FIG. 6.

### ECM Mimicking Self-Assembled Peptide Amphiphile (PA) Nanomatrix Characteristics of the Self-Assembled Peptide Amphiphile (PA):

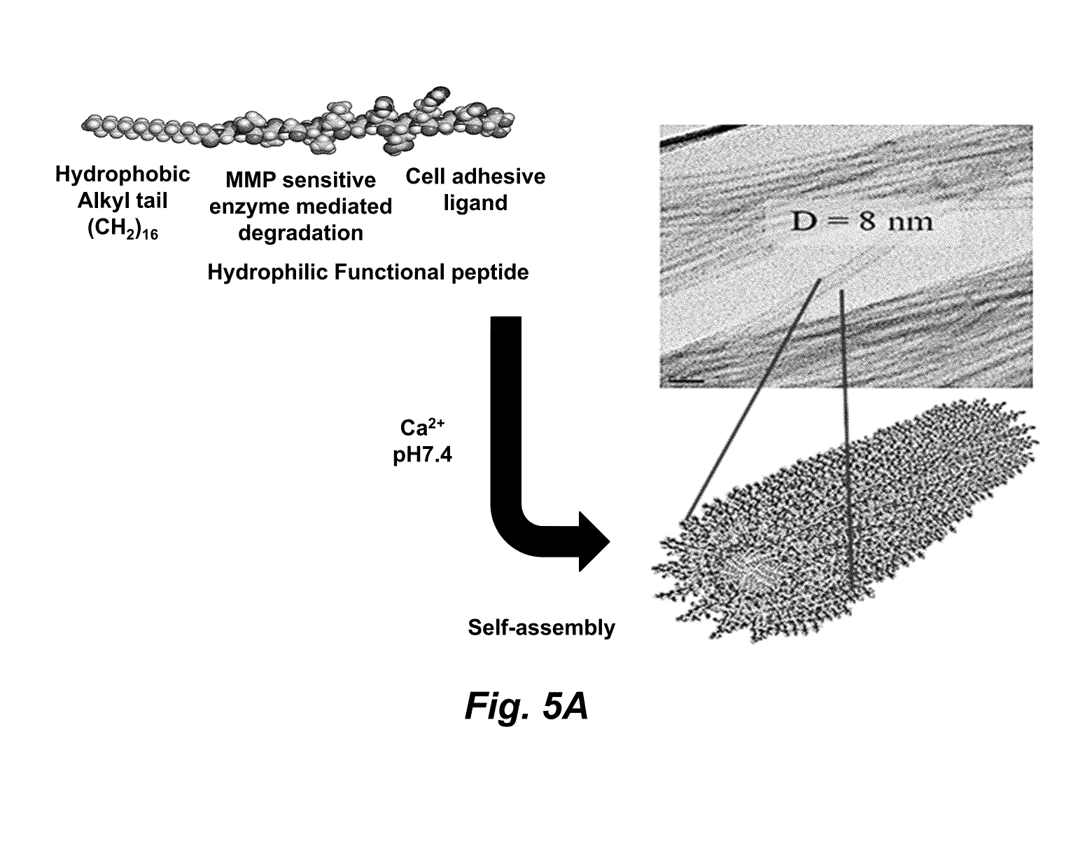

The molecular self-assembly of PAs leads to the formation of nanofibers that may be physically cross-linked to form a three-dimensional tissue-like structure by adding Ca²⁺, which initiates self-assembly, as shown in FIGS. 5A and 5B. Matrix metalloproteinase-2 enzyme degradable sequences are also included to allow cell-mediated migration through the nanomatrix, thus mimicking a characteristic property of the natural ECM. Furthermore, cell adhesive ligands may also be inscribed into the PAs to promote cell-adhesion through integrin-mediated binding.

### Transport of Isolated Pancreatic Islets Before Implantation:

Despite several promising outcomes of pancreatic islet transplantation (PIT), the necessity for multiple islet infusions has hindered the practical implementation of PIT. In practice, islet isolation and transplantation could be performed in different places, even different countries, and then shipped to the area of critical need. Additionally, severe shortages of cadaveric donors may arise in specific regions. Therefore, increasing the availability of deceased donor pancreata with islet shipment across institutions located in either domestic or internal areas should be considered to meet the current required islet numbers for successful pancreatic islet transplantation.

To increase the availability of distant clinical islet transplantations, several approaches have been introduced. To extend the cold ischemia time (CIT) required for isolating human islets from deceased donors, oxygen pre-charged perfluorodecalin has been used to minimize ischemically induced injury. Then, to ship the islets after isolation, gas-permeable bags have been used and found to improve islet recovery rates and potency after shipment. However, these are not well-established techniques, and more recent contradictory evidence has emerged. Specifically, other studies have shown that the oxygenation of explanted human pancreata using the two-layer method (TLM) can have no beneficial effect on human islets treated for prolonged CIT and commercial gas-permeable bags may not prevent anoxia that causes damage to the isolated islets during shipment.

Interestingly, compared to freshly isolated islets, islet recovery rate was higher in pre-cultured groups, indicating that the cellular stresses related to the isolation procedure still influence quality of the isolated islets, even after supplying oxygen. Considering the current under-utilization of pancreas, there remains a need for the development of a carrier that delivers human islets in a freshly-isolated condition would ensure readily available high quality islets, even after long-distance shipment. This would circumvent the problems associated with the current standards for isolating and transporting islets, which result in removal of the natural ECM environment and has restricted islet preservation advancement.

Accordingly, the disclosure further provides a cellular-level preserving method for islet shipment using an ECM-mimicking nanomatrix that forms a gel-like nanomatrix by self-assembly of peptide amphiphiles (PAs), thereby providing an ECM mimetic environment. This is facilitated through the qualities of the ECM-mimicking nanomatrix carrier: a rapid gel-like 3D network formation by self-assembly at physiological conditions for islet embedment, versatility to incorporate various cell adhesive moieties, and cell-mediated degradable sites (matrix metalloproteinase-2, MMP-2) for progressive scaffold degradation.

One aspect of the disclosure, therefore, encompasses embodiments of a biocompatible implant comprising: (i) a biocompatible nanomatrix gel comprising a plurality of a peptide amphiphile monomers cross-linked by divalent metal anions; and (ii) a biocompatible nanofiber sack, wherein said nanofiber sack is formed from a porous electrospun nanofiber sheet having crater-like surface indentations.

In embodiments of this aspect of the disclosure, the peptide amphiphile monomers can have the formula (CH₃(CH₂)₁₄CONH-GTAGLIGQERGDS) (SEQ ID NO.: 1).

In embodiments of this aspect of the disclosure, the biocompatible implant can further comprise at least one cell growth factor, wherein the at least one cell growth factor can be incorporated in the nanomatrix gel, incorporated in the nanofiber sack, or both incorporated in the nanomatrix gel and in the nanofiber sack.

In embodiments of this aspect of the disclosure, the biocompatible implant can further comprise a population of isolated animal or human cells embedded in the nanomatrix gel.

In embodiments of this aspect of the disclosure, the at least one cell growth factor can be releasable from the biocompatible implant.

In embodiments of this aspect of the disclosure, the at least one cell growth factor can be an angiogenic factor that can induce the formation of a blood vessel when the biocompatible implant is implanted in a recipient animal or human subject.

In embodiments of this aspect of the disclosure, the population of isolated animal or human cells embedded in the gel can be a pancreatic islet or a population of pancreatic islets.

In embodiments of this aspect of the disclosure, the pancreatic islet or islets can be isolated from an animal or human, or a cultured islet or islets.

In embodiments of this aspect of the disclosure, the polymer nanofibers forming the nanofiber sheet can comprise poly-ε-caprolactone.

In embodiments of this aspect of the disclosure, the nanofiber sheet can further comprise at least one cell growth factor.

In embodiments of this aspect of the disclosure, the at least one cell growth factor embedded in the nanofiber sheet, attached to an outer surface thereof, or both embedded in the nanofiber sheet and attached to an outer surface thereof.

In embodiments of this aspect of the disclosure, the at least one cell growth factor is releasable from the implant in a multi-step process.

Another aspect of the disclosure encompasses embodiments of a biocompatible electrospun nanofiber sheet, wherein said sheet is porous and comprises a plurality of crater-like indentations on at least one surface of said nanofiber sheet.

In embodiments of this aspect of the disclosure, the polymer nanofibers forming the nanofiber sheet can comprise poly-ε-caprolactone.

In embodiments of this aspect of the disclosure, the biocompatible nanofiber sheet can further comprise at least one cell growth factor.

In embodiments of this aspect of the disclosure, the at least one cell growth factor can be embedded in the nanofiber sheet, attached to an outer surface thereof, or both embedded in the nanofiber sheet and attached to an outer surface thereof.

In embodiments of this aspect of the disclosure, the the at least one cell growth factor can be releasable from the nanofiber sack.

In some embodiments of this aspect of the disclosure, at least one cell growth factor is an angiogenic factor that can induce the formation of a blood vessel when the biocompatible implant is implanted in a recipient animal or human subject.

Still another aspect of the disclosure encompasses embodiments of a method of manufacturing a biocompatible nanofiber sheet, the method comprising the steps of: (i) electrospinning a biocompatible polymer onto a collector to form a nanofiber sheet, wherein the biocompatible polymer is co-delivered to the collector with a plurality of leachable particles; and (ii) contacting the electrospun nanofiber sheet with a composition capable of removing the particles from the nanofiber sheet, thereby generating a porous nanofiber sheet having crater-like indentations in at least one surface of the nanofiber sheet.

In embodiments of this aspect of the disclosure, the biocompatible polymer can be poly-ε-caprolactone.

In embodiments of this aspect of the disclosure, the biocompatible polymer can be delivered to the collector at a flow rate from about 0.5 ml/h to about 5.0 ml/h, at a distance from about 10 cm to about 30 cm, a voltage from about 10 to about 25 kV and for about 30 min to about 3 h.

In embodiments of this aspect of the disclosure, the method can further comprise the step of contacting the nanofiber sheet with at least one cell growth factor desired to be incorporated into the nanofiber sheet.

In some embodiments of this aspect of the disclosure, the leachable particles can be particles of a carbonate or a bicarbonate, and wherein the composition capable of removing the particles from the nanofiber sheet is an acid, thereby generating a gas that generates the crater-like indentations.

Still yet another aspect of the disclosure encompasses embodiments of a method of maintaining a population of isolated animal cells in a state suitable for implantation into a recipient animal or human subject, the method comprising the steps of (i) embedding a population of cells or cell aggregates thereof, in an implantable biomimetic nanomatrix gel comprising: (a) a plurality of a peptide amphiphile monomers cross-linked by divalent metal anions; and (b) at least one cell growth factor; (ii) encapsulating the nanomatrix gel in a nanofiber sack, wherein said nanofiber sack is formed from a nanofiber sheet manufactured by electrospinning a biocompatible polymer; and (iii) maintaining the encapsulated nanomatrix under conditions substantially allowing the population of cells or cell aggregates thereof to retain viability and their biological function.

In embodiments of this aspect of the disclosure, the cell aggregates are pancreatic islets.

In embodiments of this aspect of the disclosure, the biocompatible polymer forming the nanofiber sheet is poly-ε-caprolactone.

In embodiments of this aspect of the disclosure, the nanofiber sack is porous and includes a plurality of crater-like indentations in an outer surface of the nanofiber sack.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

### Materials and Animals

CMRL-1066 tissue culture medium, L-glutamine, penicillin, and streptomycin were purchased from Gibco (Grand Island, N.Y.). Fetal bovine serum was obtained from Hyclone (Logan, Utah). Male Sprague-Dawley rats (250-300 g) were from Harlan Laboratories (Indianapolis, Ind.). Collagenase type XI was from Sigma Chemical Co. (St. Louis, Mo.). Rat insulin ELISA kit was from Crystal Chem Inc. (Downers Grove, Ill.).

### Example 2

### Islet Isolation and Culture

Pancreatic islets were isolated from male Sprague-Dawley rats (250-300 g) by collagenase digestion as described in McDaniel et al., (1983) Methods Enzymol. 98: 182, incorporated herein by reference in its entirety. After digestion, islets were isolated by density gradient purification and individually selected under a dissection microscope. Fifty isolated islets were used per sample for each condition group. All islet samples were cultured at 37° C. in an atmosphere of 95% air and 5% CO₂ in CMRL-1066 tissue culture medium supplemented with 2 mM L-glutamine, 10% heat-inactivated fetal bovine serum, 100 U/mL penicillin, and 100 μg/mL streptomycin.

In contrast to the traditional islet culturing methods, which lead to variability in the observed results due to physical loss of islets during cultivation, a modified insert chamber was devised to more accurately measure islet function. In traditional culture methods, islets are susceptible to necrotic death within their central cores due to islet size, non-proliferative nature, and the variability of culture conditions. The devised insert chamber provided a consistent islet culturing method that not only measured islet function more accurately, but also provided a better system for quantifying the viability of the remaining islets over long-term cultivation.

### Example 3

### Synthesis of Peptide Amphiphiles (PA)

Using standard Fmoc-chemistry, the peptide amino acid sequence GTAGLIGQERGDS (SEQ ID No.: 1) was synthesized on an Advanced Chemtech Apex 396 peptide synthesizer as described by Anderson et al., (2009) Biomacromolecules 10: 2935; Anderson et al., (2009) ACS Nano. 3: 3447; and Kushwaha et al., (2010) Biomaterials 31: 1502, all incorporated herein by reference in their entireties. After synthesis, the peptide was alkylated at the N-termini with palmitic acid by a manual coupling reaction. Alkylation was performed for 24 h at room temperature in a mixture of o-benzotriazole-N,N,N′,N′-tetramethyluroniumhexafluorophosphate, diisopropylethylamine, and dimethylformamide. Cleavage and deprotection were performed with a mixture of trifluoroacetic acid, deionized water, triisopropylsilane, and anisole (40:1:1:1) for 3 h at room temperature. The PA solution was precipitated in cold ether after removing excess trifluoroacetic acid and lyophilized. Matrix-assisted laser desorption ionization time of flight mass spectrometry was used for PA characterization.

### Example 4

### Islet Encapsulation within PA Self-Assembled Nanomatrix Gels

PA stock solution (2% weight/volume) was prepared and buffered to a pH of about 7.0 with NaOH. Self-assembly of the PA with rat islets was induced by combining 50 μL of PA solution with 50 μL of complete CMRL-1066 medium and 15 μL 0.1 M CaCl₂ in 12-well silicone flexiPERM cell-culture chambers (Sigma-Aldrich, St. Louis, Mo.) attached to glass coverslips. The molar ratio between PA and calcium ion (Mr=Ca²⁺/PA) was held constant at an Mr of 2, as described by Jun et al., (2005) Adv. Mater. 17: 2612, incorporated herein by reference in its entirety. The PA self-assembled nanomatrix gel was formed as a sphere-shaped hydrogel with an approximate 7.2 mm diameter. After encapsulating, the PA self-assembled nanomatrix gel containing 50 hand-picked rat islets (nanomatrix group) was transferred into a fabricated mesh according to the disclosure and cultured in a 12-well non-tissue culture-treated plate (Corning Costar, Corning, N.Y.).

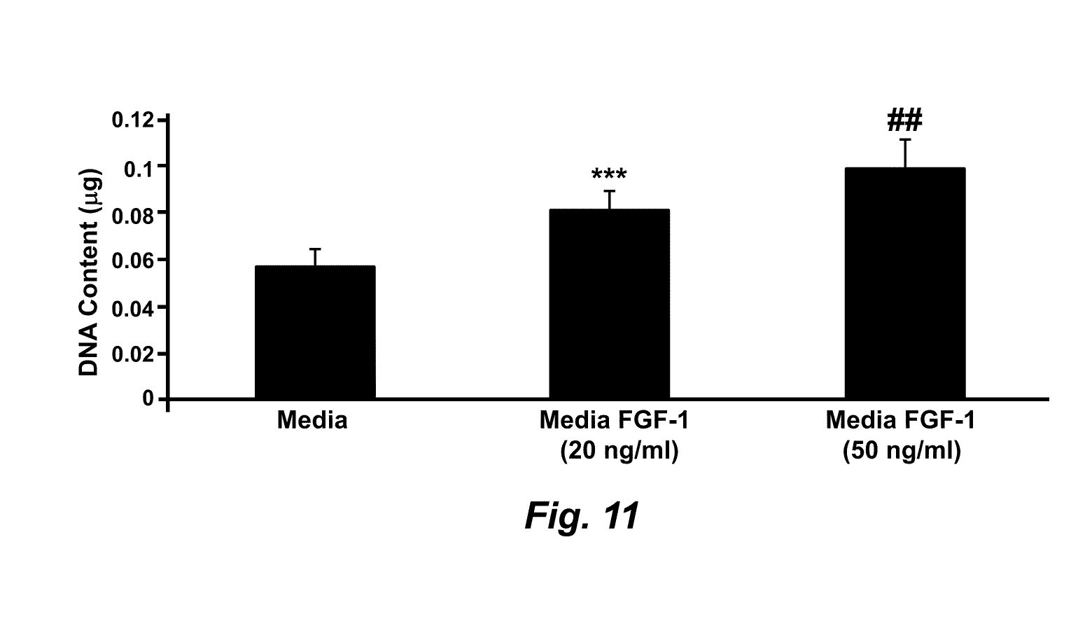

To evaluate the effect of the PA self-assembled nanomatrix gel on isolated rat islet function, three groups were designed: (i) bare group: isolated rat islets cultured in a 12-well non-tissue culture-treated plate; (ii) insert group: isolated rat islets cultured in modified insert chambers; (iii) nanomatrix group: isolated rat islets encapsulated within the PA self-assembled nanomatrix gel and cultured in modified insert chambers. In the modified insert chamber design, two factors were emphasized: (a) maintaining the islet number throughout the culture period because periodic medium change could lead to loss of islets and (b) accurately quantifying the secreted insulin of each experimental group with little variance. From using traditional culture methods, it was found that most rat islets remained weakly attached to the culture surface, leading to some physical loss of islets when replacing medium over long-term culture. As a result, relative variations in the remaining islets during the cultivation could affect the assessment of islet function. Thus, in this study, a modified insert chamber, as shown in FIG. 12, in which a 5 μm nylon mesh sheet was placed into a commercial insert chamber was developed to retain free-floating islets, thereby preventing physical loss of islets.

### Example 5

### Glucose-Stimulated Insulin Secretion

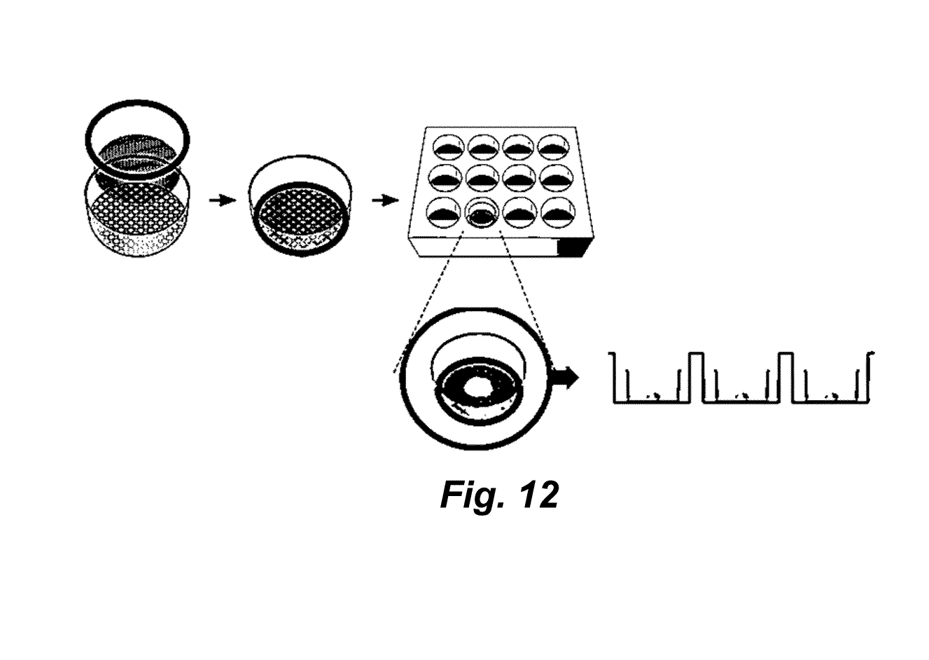

Glucose-stimulated insulin secretion was assessed at 3, 7, and 14 days after encapsulation. To eliminate any residual insulin, all samples were pre-incubated for 1 h in low-glucose Krebs-Ringer bicarbonate buffer (low-glucose KRB) (25 mM HEPES, 115 mM NaCl, 24 mM NaHCO₃, 5 mM KC, 1 mM MgCl₂, 2.5 mM CaCl₂, and 0.1% bovine serum albumin, and 3 mM D-glucose, pH 7.4). Then, each sample was placed in 1 mL low-glucose KRB for 1 h, followed by incubation in 1 mL high-glucose KRB (20 mM D-glucose) for 1 h. The supernatant was withdrawn, and insulin was measured by the ELISA method. To normalize the secreted insulin data, as shown in FIG. 13, a fluorometric PicoGreen DNA kit (Molecular Probes, Eugene, Oreg.) was used to measure DNA content of each sample using a microplate fluorescent reader (Synergy HT; BIO-TEK Instrument, Winooski, Vt.).

### Assessment of Glucose-Stimulated Insulin Secretion:

After a period of cultivation, glucose-stimulated insulin secretion responses were measured to evaluate the function of encapsulated rat islets. Over the 14 days of cultivation, both the bare and insert groups showed a marked decrease in insulin secretion, whereas the nanomatrix group maintained glucose-stimulated insulin secretion, even after 14 days. Additionally, islets in the bare group were not responsive to elevated levels of glucose after 14 days, as most of the islets not only lost their functionality, but were found to be completely missing due to the periodic medium changes. In contrast, the response of β-cells to the high-glucose condition was maintained throughout for the nanomatrix group. Further, over the entire cultivation period, there was a significant statistical difference in glucose-stimulated insulin responses for the nanomatrix group. After 3, 7, and 14 days, the low glucose response values were 8.7±9.6, 19.9±11.2, and 7.0±1.9 ng, whereas the high glucose response values were 145.1±49.8, 118.3±71.0, and 105.6±52.5 ng, respectively.

These results were validated by the stimulation index (SI) data observed. To compare the insulin secretion values between groups, each average SI obtained by dividing average high glucose response value with average low glucose response value of each group was calculated. The average SI values were 16.7, 5.9, and 15.0 for islets in the nanomatrix group after 3, 7, and 14 days, respectively. However, the average SI values over the same time points were 2.0, 1.8, and 1.6 for the insert groups and 1.6, 1.9, and 1.0 for the bare groups, respectively. Thus, the average SI values after 14 days for the nanomatrix group were approximately seven-fold more than the insert group and almost nine-fold greater than the bare group, as shown in FIGS. 14-16.

### Example 6

### Islet Viability Assessment

Islet cell viability in each group was assessed by microscopic examination using fluorescein diacetate/propidium iodide (FDA/PI) staining at 3, 7, and 14 days after encapsulation. An FDA stock solution was prepared by dissolving 10 mg FDA into 2 mL of acetone. The FDA stock solution was stored at −20° C. When in use, 10 μL of FDA stock solution was diluted with 990 μL of phosphate-buffered saline (PBS). PI (1 mg/mL; Invitrogen, Eugene, Oreg.) was prepared each time to be used immediately, as 50 μL of solution was diluted with 450 μL of PBS. For viability staining, each sample was immersed in a mixture of 2 mL PBS, 10 μL of diluted PI, and 20 μL of diluted FDA. Stained islets were observed with a fluorescence microscope equipped with a high-pressure mercury arc lamp. Fluorescein dyes that deacetylated from FDA through non-specific esterases in the cytoplasm of cells were shown under a fluorescent blue filter, whereas PI dyes, staining nucleic acids of dead cells, were viewed under a fluorescent green filter.

### FDA/PI Staining to Determine Islet Viability:

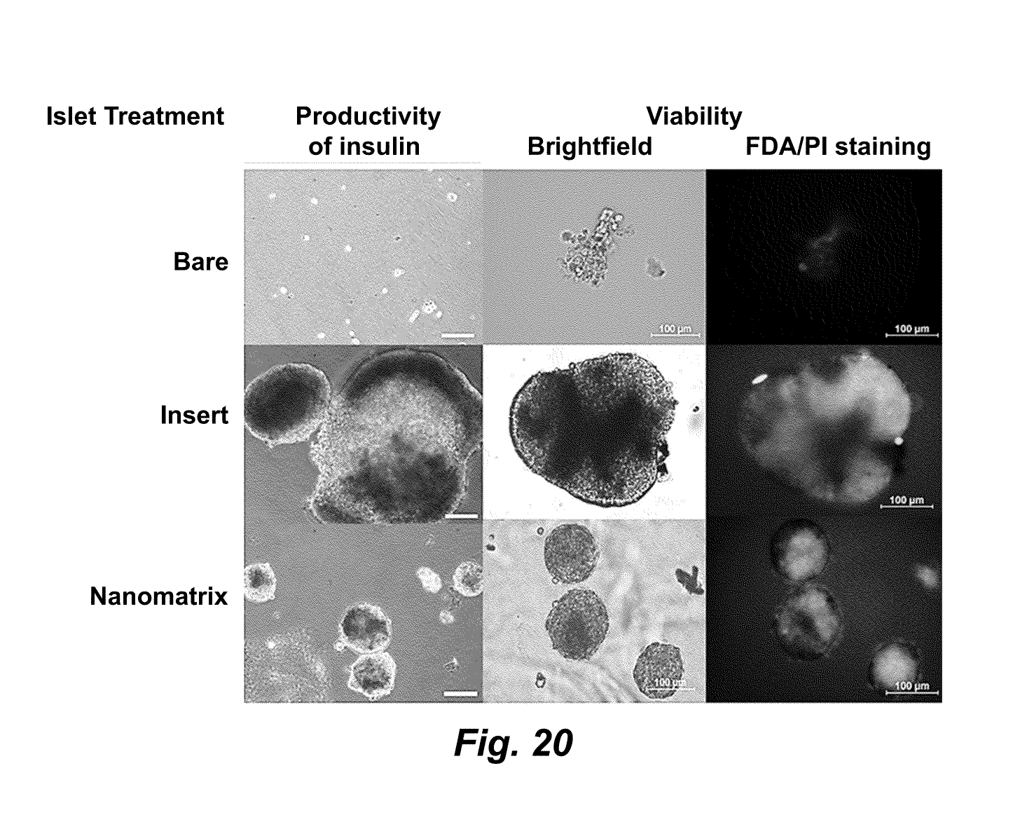

After 3 days, islets in all three groups (bare, insert, nanomatrix embedded) displayed maintained viability. After 7 days, the cores of the islets in the bare and insert groups developed necrotic cores (dark centers localized with red fluorescence), whereas the islets in the nanomatrix group still retained most of their viability, as shown in FIG. 19. After 14 days, as shown in FIG. 20, in the insert or bare groups, there were fewer remaining islets in general, and out of the retained islets, many were fragmented opaque clusters of islets or simply cellular debris lacking in viability. Conversely, the islets in the nanomatrix group still maintained islet integrity and almost all remained viable. These FDA/PI staining results represent a marked improvement in maintained islet viability for the nanomatrix group over the entire incubation period compared to the other two groups, which began to display necrosis and reduced viability after 7 days.

### Example 7

### Evaluation of Insulin-Producing β-Cells Using Dithizone Staining

To identify insulin-producing β-cells from each group, dithizone (DTZ) staining was used after 3, 7, and 14 days post-encapsulation. DTZ forms a red-colored complex when reacted with zinc, indicating positive staining for insulin production. To make the DTZ stock solution, 50 mg of DTZ was dissolved in 5 mL of dimethyl sulfoxide and diluted with 30 mL of PBS. The stock solution was filtered with a 0.45 μm filter. DTZ solution was added to each group for microscopic examination at the predetermined time points.

### DTZ Staining to Evaluate Insulin-Producing β-Cells:

DTZ staining used to qualitatively assess the function of the islets appeared as a crimson red-positive stain in the insulin-producing β-cells within the islets (Latif et al., (1988) Transplantation 45: 827). After 3 days, all three groups (bare, insert, nanomatrix embedded) showed positive DTZ staining. After 7 days, the bare and insert groups had significantly reduced positive DTZ staining, whereas the nanomatrix group still maintained high positive staining. After 14 days, the bare group had no intact islets left to stain, and the insert group only retained positive staining in the peripheral areas of a few disintegrated islets. In contrast, the islets in the nanomatrix group still maintained integrity and DTZ-positive staining throughout the core of the islets. These results, as shown in FIG. 18, indicate that the nanomatrix group maintained function throughout the 14 days.

### Example 8

### Statistical Analysis

All experiments were performed at least three independent times in quadruplicate. All values were denoted as means±standard deviation. Statistical analysis was performed using SPSS 15.0 software (SPSS, Inc., Chicago, Ill.). One-way analysis of variance was used for statistical comparison. A level p<0.05 was considered to be statistically significant.

### Example 9

### Electrospinning Cotton Ball-Like Electrospun Scaffolds

Similar to traditional electrospinning, PCL pellets were dissolved at a ratio of 75 mg/ml in a solvent solution of 1:1 (v: v) chloroform and methanol and transferred to a syringe chamber. The filled syringe fitted with a 25 gauge blunt-tipped needle nozzle was then placed into a syringe pump with a set flow rate of 2.0 ml/h and at a distance of 15 cm from the front plane of the collector. The nozzle was attached to the positive terminal of a high voltage generator through which a voltage of +15 kV was applied 1 mm from the needle opening, and the three dimensional electrospun scaffold was fabricated onto a custom-made collector.

The collector for the cotton ball-like electrospun scaffolds was crafted by embedding an array of 1.5 inch long stainless steel probes in a spherical foam dish (diameter: 8 in., shell thickness: 0.125 in.; Fibre Craft, Niles, Ill.) backed by a stainless steel lining to provide an electrical ground. The needles were placed at 2 inch intervals radiating from the center of the dish in five equidistant directions. The nanofibers were allowed to accumulate throughout the electrospinning process and then removed with a glass rod.

### Example 10

### Scaffold Characterization-Scanning Electron Microscope (SEM) Imaging

The ePCL scaffolds were mounted on an aluminum stub and sputter coated with gold and palladium. A Philips SEM 510 (FEI, Hillsboro, Oreg.) at an accelerating voltage of 20 kV was used to image the scaffolds, and the fiber diameters were measured using GIMP 2.6 for Windows.

### Example 11

### Confocal Microscope Imaging

To visually contrast nanofiber network organization in the traditional flat-plate electrospun scaffold with the cotton ball-like electrospun scaffold, scaffolds were incubated in 4′,6-diamidino-2-phenylindole (DAPI; Invitrogen, Carlsbad, Calif.) for 4 h. Scaffolds were then imaged using a Zeiss LSM 710 Confocal Laser Scanning Microscope (Thornwood, N.Y.) and analyzed using Zen 2009 software. Since DAPI is strongly attracted to the hydrophobic PCL, the fluorescence clearly illuminated the nanofibrous structures of the scaffolds.

### Example 12

### Renal Subcapsular Islet Transplantation in a Diabetic Rat Model

For the islets only group, STZ-induced diabetic rats received 1500 syngeneic islets through left renal subcapsular route. For the islets embedded within the nanomatrix gel group, a mixture of 1500 syngeneic islets and the nanomatrix solution was injected directly into the left renal subcapsule of STZ-induced diabetic rats and immediately formed into the nanomatrix gel.

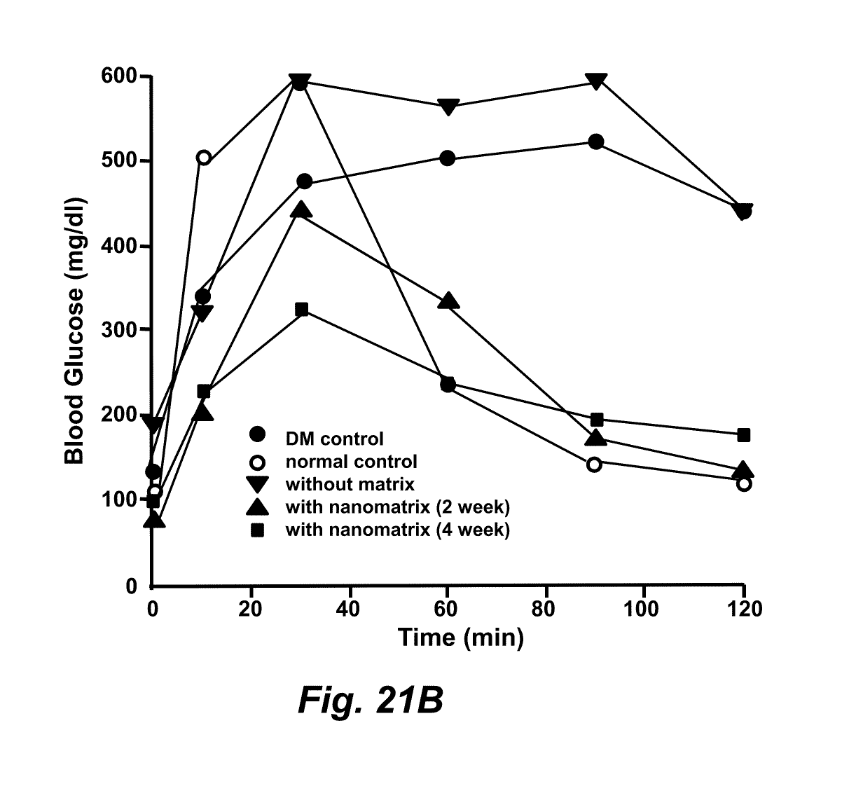

The non-fasting blood glucose (NFBG) stayed at a high level in the islets-only group. In contrast, in the islets embedded within the nanomatrix gel group, the NFBG became normal after 4 days and then leveled off for 1 week. Although the NFBG rebounded to hyperglycemia after 2 weeks, the fasting blood glucose level remained at a normal state for 28 days, as shown in FIG. 21A. In addition, the dramatic rebounce of NFBG was observed after removal of graft-bearing kidney in the islets embedded within the nanomatrix gel group.

An intraperitoneal glucose tolerance test (IPGTT) of the islets-only group showed the same pattern as the diabetic control. In the islets embedded within the nanomatrix gel group, the results of IPGTT performed at 2 and 4 weeks indicated a similar pattern to the normal control, as shown in FIG. 21B. Accordingly, the nanomatrix gel of the disclosure offers an model for improving the engraftment of syngeneic islets in the kidney capsule by creating a nurturing and protective microenvironment for islets.

### Example 13

### Human Islets Encapsulated within the Nanomatrix Gel

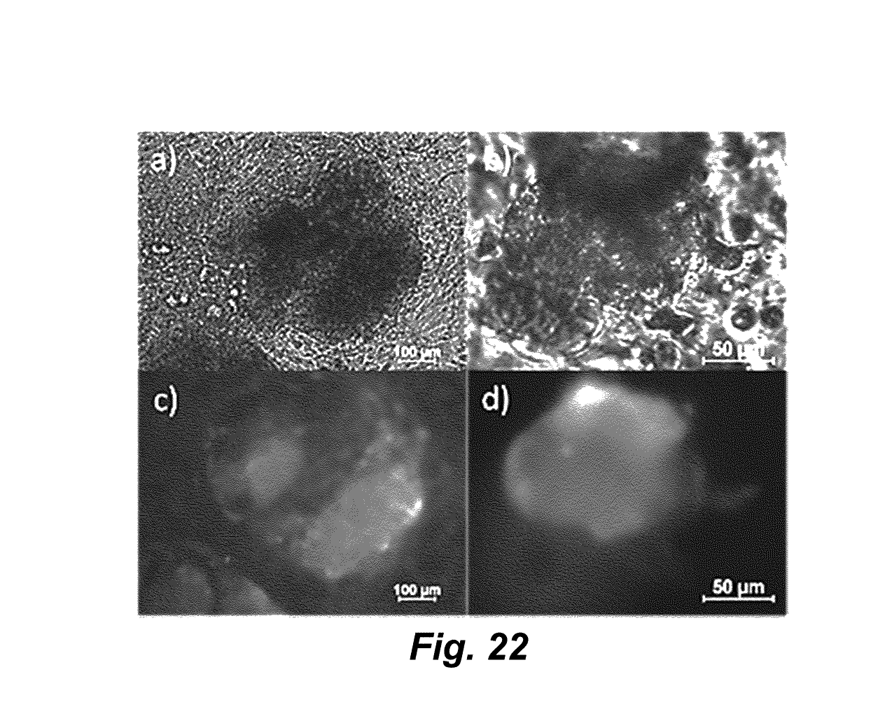

The efficacy of the nanomatix for encapsulating isolated human islets was investigated. Human islets were embedded in the nanomatrix gel and compared with a control group. After 14 days, the isolated human islets kept their integrity with good viability, shown in the FDA/PI staining (FIG. 22, Panels (b) and (d)), whereas the human islets without the nanomatrix gel slowly disintegrated (FIG. 22, Panels (a) and (c)). These results indicate that the nanomatrix gel supports isolated human islet integrity and viability.

### Example 14

### Recovery of Islet ECM Microenvironment Using a PA Nanomatrix Gel

The islet ECM microenvironment can be recovered using a peptide amphiphile (PA) nanomatrix gel according to the disclosure. The PA according to the disclosure is advantageously composed of a hydrophobic alkyl tail attached to hydrophilic functional peptide sequences, as shown in FIG. 5A. This amphiphilic design is advantageous for inducing the self-assembly of the PA into a nanomatrix gel by addition of calcium ions, as shown in FIGS. 5A and 5B.

One advantageous, but not limiting, embodiment of the PA useful in forming the nanomatrix gel of the disclosure is PA-RGDS that has the structure (CH₃(CH₂)₁₄CONH-GTAGLIGQERGDS) (SEQ ID NO.: 1) synthesized using standard Fmoc-chemistry on an Advanced Chemtech Apex 396 peptide synthesizer. PA-RGDS, accordingly, consists of a hydrophobic alkyl tail, the cell an enzyme-mediated degradable site specific for MMP-2, and adhesive ligand RGDS.

### Example 15

Electrospinning methods and apparatus suitable for use in the generation of the biocompatible implants of the disclosure are described, for example, in U.S. patent application Ser. No. 13/081,820, incorporated herein by reference in its entirety. For example, poly-ε-caprolactone (7 to 20 wt. %) (PCL, Mₙ: 80,000; Sigma Aldrich, St. Louis, Mo.) solution was prepared using various solvent solutions, such as 1:1 (v:v) chloroform: methanol; 1:1 (v:v) dimethylformamide: dichloromethane; or trifluoroethanol. The prepared PCL solution was loaded in a syringe fitted with a 25 gauge blunt-tipped needle. The syringe was placed into a syringe pump (KD Scientific, Holliston, Mass.) with a flow rate ranging from about 0.5 ml/h to 5.0 ml/h, and a distance ranging from about 10 cm to about 30 cm between the needle tip and the front plane of an aluminum foil collector. Electrospinning was carried out at a voltage ranging from about 10 kV to about 25 kV using a high-voltage generator (Gamma High-Voltage Research, Ormond Beach, Fla.) for about 30 min to about 3 h to fabricate an electrospun poly-ε-caprolactone (ePCL) scaffold.

Various collectors can be used to collect ePCL nanofibers such as metal plates, an array of metal probes embedded in non-conductive dishes, and also with wet conditions such as is shown in FIG. 23.

During an electrospinning process, sodium bicarbonate particles with the size from about 100 μm to about 300 μm were introduced into the Taylor Cone as the ePCL nanofibers were being formed. Various weight ratios of PCL: sodium bicarbonate (1:1, 1:4, 1:7, 1:10, 1:13, 1:16, and 1:20) were used to control the size and distribution of crater like structures on the ePCL scaffold. The total sodium bicarbonate needed for each ratio was measured based on the determined time period, and divided into small amount for each allotment in every 5 min. Each allotment was added for 60 seconds in 5 min intervals.

After electrospinning, the sodium bicarbonate-containing ePCL nanofiber scaffold was immersed in a 50% citric acid solution for 1 h to generate carbon dioxide (CO₂) and then in deionized water for 3 days to dissolve remaining sodium bicarbonate. The deionized water was replaced every day, and the samples were dried at room temperature.

### Example 16

### Fabrication of a Hybrid Nanosack

The hybrid nanosack was fabricated by combining a self-assembled PA nanomatrix gel with an ePCL nanofiber sheet with crater-like structures, as shown in FIGS. 6-7C. Six PAs were synthesized with different cell adhesive ligands (PA-RGDS, PA-YIGSR, PA-DGEA, PA-IKLLI, PA-IKVAV, and PA-S (no cell adhesive ligand as a negative control)) using standard Fmoc-chemistry on an Advanced Chemtech Apex 396 peptide synthesizer as described, for example, in Anderson et al., (2011) Acta Biomaterialia 7: 675-682, incorporated herein by reference in its entirety. All PAs consisted of a cell adhesive ligand, an enzyme-mediated degradable site (GTAGLIGQ) (SEQ ID NO.: 2) specific for MMP-2, and a hydrophobic alkyl tail attached to the N-terminus of the peptide segment. Self-assembly of each nanomatrix gel was induced by the addition of CaCl₂. The hybrid nanosack was then fabricated by wrapping the nanomatrix gel with an ePCL nanofiber sheet with crater-like structures, as shown in FIGS. 6-7C.

### Example 17

### Multi-Stage FGF Release Kinetics from the Hybrid Nanosack

The multi-stage release kinetic of FGF (in this case, FGF-1) from the hybrid nanosack was demonstrated, as shown in FIG. 3. FGF-1 (100 ng) was used to evaluate FGF-release kinetics, and three different conditions were designed for 14 days: Group A, 100 ng of FGF-1 coated on the ePCL nanofiber sheet; Group B, 100 ng of FGF-1 entrapped within the nanomatrix gel; and Group C, the hybrid nanosack consisting of 50 ng of FGF-1 entrapped within the nanomatrix gel and wrapped with the ePCL nanofiber sheet coated with 50 ng of FGF-1.

Group A showed a rapid burst release in 14 h (approximately 80%) with very little additional release. Group B showed a very slow release over 14 days (approximately 20%). However, Group C presented an initial burst release of FGF-1 in first 24 hours (approximately 40%) followed by a sustained release over a period of 14 days (approximately 60%). These results showed that the hybrid nanosack can be capable of multi-stage releases of FGF-1 for both de novo angiogenesis and the formation of a stable vascular network.

### Example 18

### Implantation of the FGF-2 Treated Hybrid Nanosack into the Omentum without Islets and Evaluation of Revascularization

Data show an FGF-2-treated hybrid nanosack (50 ng FGF-2 entrapped in the PA-RGDS nanomatrix gel and 50 ng FGF-2 surface-coated on the ePCL nanofiber sheet) resulted in revascularization around the omentum of a rat after 2 weeks, as shown in FIG. 7C. Notably, micro-computer tomography (μ-CT) image clearly showed that porous crater-like structures allow infiltration of blood vessels into the hybrid nanosack, as shown in FIG. 7B.

What is claimed:

1. A biocompatible implant comprising:
(i) a biocompatible nanomatrix gel comprising a plurality of a peptide amphiphile monomers cross-linked by divalent metal anions; and
(ii) a biocompatible nanofiber sack, wherein said nanofiber sack is formed from a porous electrospun nanofiber sheet having crater-like surface indentations.

2. The biocompatible implant according to claim 1, wherein the peptide amphiphile monomers have the formula (CH₃(CH₂)₁₄CONH-GTAGLIGQERGDS) (SEQ ID NO.: 1).

3. The biocompatible implant according to claim 1, further comprising at least one cell growth factor, wherein the at least one cell growth factor is incorporated in the nanomatrix gel, is incorporated in the nanofiber sack, or both incorporated in the nanomatrix gel and in the nanofiber sack.

4. The biocompatible implant according to claim 1, further comprising a population of isolated animal or human cells embedded in the nanomatrix gel.

5. The biocompatible implant according to claim 3, wherein the at least one cell growth factor is releasable from the biocompatible implant.

6. The biocompatible implant according to claim 3, wherein the at least one cell growth factor is an angiogenic factor that can induce the formation of a blood vessel when the biocompatible implant is implanted in a recipient animal or human subject.

7. The biocompatible implant according to claim 4, wherein the population of isolatanimal or human cells embedded in the gel is a pancreatic islet or a population of pancreatic islets.

8. (canceled)

9. The biocompatible implant according to claim 1, wherein the polymer nanofibers forming the nanofiber sheet comprise poly-ε-caprolactone.

10. The biocompatible implant according to claim 3, wherein the nanofiber sheet further comprises at least one cell growth factor, and wherein the at least one cell growth factor is embedded in the nanofiber sheet, attached to an outer surface thereof, or both embedded in the nanofiber sheet and attached to an outer surface thereof.

11. (canceled)

12. The biocompatible implant according to claim 3, wherein the at least one cell growth factor is releasable from the implant in a multi-step process.

13. A biocompatible electrospun nanofiber sheet, wherein said sheet is porous and comprises a plurality of crater-like indentations on at least one surface of said nanofiber sheet.

14. The biocompatible nanofiber sheet according to claim 13, wherein the polymer nanofibers forming the nanofiber sheet comprise poly-ε-caprolactone.

15. The biocompatible nanofiber sheet according to claim 13, further comprising at least one cell growth factor, wherein the at least one cell growth factor is embedded in the nanofiber sheet, attached to an outer surface thereof, or both embedded in the nanofiber sheet and attached to an outer surface thereof.

16. (canceled)

17. The biocompatible nanofiber sheet according to claim 15, wherein the at least one cell growth factor is releasable from the nanofiber sack.

18. The biocompatible nanofiber sheet according to claim 15, wherein the at least one cell growth factor is an angiogenic factor that can induce the formation of a blood vessel when the biocompatible implant is implanted in a recipient animal or human subject.

19. A method of manufacturing a biocompatible nanofiber sheet comprising the steps of:
(i) electrospinning a biocompatible polymer onto a collector to form a nanofiber sheet, wherein the biocompatible polymer is co-delivered to the collector with a plurality of leachable particles; and
(ii) contacting the electrospun nanofiber sheet with a composition capable of removing the particles from the nanofiber sheet, thereby generating a porous nanofiber sheet having crater-like indentations in at least one surface of the nanofiber sheet.

20. (canceled)

21. (canceled)

22. The method according to claim 19, further comprising the step of contacting the nanofiber sheet with a composition comprising at least one cell growth factor desired to be incorporated into the nanofiber sheet.

23. (canceled)

24. A method of maintaining a population of isolated animal cells in a state suitable for implantation into a recipient animal or human subject, the method comprising the steps of
(i) embedding a population of cells or cell aggregates thereof, in an implantable biomimetic nanomatrix gel comprising:
(a) a plurality of a peptide amphiphile monomers cross-linked by divalent metal anions; and
(b) at least one cell growth factor;
(ii) encapsulating the nanomatrix gel in a nanofiber sack, wherein said nanofiber sack is formed from a nanofiber sheet manufactured by electrospinning a biocompatible polymer; and
(iii) maintaining the encapsulated nanomatrix under conditions substantially allowing the population of cells or cell aggregates thereof to retain viability and their biological function.

25. (canceled)

26. (canceled)

27. (canceled)


SEQUENCE LISTING (2 sequences)

SEQ ID NO: 1 - protein, 13 aa, Artificial sequence
  Peptide amphiphile having C18 alkyl chain at N-terminus
GTAGLIGQERGDS

SEQ ID NO: 2 - protein, 8 aa, Artificial sequence
  Metalloproteinase-2 (MMP-2) degradable site
GTAGLIGQ